(19) United States
(12) Patent Application Publication
(10) Pub. No.: US 2021/0125691 A1
(43) Pub. Date: Apr. 29, 2021

(54) SYSTEMS AND METHOD FOR DESIGNING ORGANIC SYNTHESIS PATHWAYS FOR DESIRED ORGANIC MOLECULES

(71) Applicant: Molecule One sp. z o.o.

(72) Inventors: Pawel Wlodarczyk-Pruszynski, Warszawa (PL); Piotr Byrski, Warszawa (PL); Pawel Laskarzewski, Warszawa (PL); Mikolaj Sacha, Krakow (PL); Mikolaj Blaz, Musuly (PL); Szymon Pilkowski, Zrebice Pierwsze (PL); Mateusz Bruno-Kaminski, Krakow (PL); Stanislaw Jastrzebski, Warszawa (PL)

(73) Assignee: Molecule One sp. z o.o.,

(21) Appl. No.: 17/060,765

(22) Filed: Oct. 1, 2020

Related U.S. Application Data
(60) Provisional application No. 62/909,160, filed on Oct. 1, 2019.

(51) Int. Cl. G16C 20/10; G16C 20/70; G06N 20/00; G05B 19/4155

(52) U.S. Cl. CPC G16C 20/10; G16C 20/70; G05B 2219/32287; G05B 19/4155; G06N 20/00

(57) ABSTRACT

Methods and systems provide proposed pathways for synthesizing chemical reactions given a user-proposed target molecule, user-provided reaction constraints, or a combination of both. Embodiments may leverage training the model using both known successful reactions and infeasible reactions, either known or created by a prior use of the model. Chemical reactions for producing the target molecule and substrates are proposed using the model. From the proposed reactions, synthesis pathways are extracted and ranked according to a cost estimation. The ranked synthesis pathways are then provided to the user.

### CROSS REFERENCE TO RELATED CASES

This application claims priority to U.S. Provisional Patent Application No. 62/909,160, entitled “SYSTEMS AND METHOD FOR DESIGNING ORGANIC SYNTHESIS PATHWAYS FOR DESIRED ORGANIC MOLECULES,” filed Oct. 1, 2019, which is incorporated in its entirety.

### TECHNICAL FIELD

The claimed subject matter relates generally to the field of chemical synthesis and more specifically to methods for automating the determination and display of chemical synthesis pathways.

### BACKGROUND

Typically, for each drug that makes it to the market, as many as 20 thousand drug-like molecules need to be made in a laboratory and tested. The molecule-making process is called chemical synthesis. The task in a retrosynthesis is to find substrates that react to yield a target molecule. Determining how to synthesize a molecule is highly inefficient and prone to errors. It involves chemists manually reviewing tens or hundreds of scientific papers. Chemical synthesis is the overlooked bottleneck in drug discovery.

Thus, what is needed is a method and system that speeds up or even automates the determination of synthesis pathways.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The embodiments are illustrated by way of example and not limitation in the figures of the accompanying drawings, in which like references indicate similar elements, and in which:

FIG. 1 is a flow chart of an embodiment of a method for proposing a synthesis pathway;

FIG. 2 is a flow chart of steps in an embodiment of elements of a method for proposing a synthesis pathway;

FIG. 3 is flow chart of an embodiment of a method 300 for proposing a synthesis pathway;

FIG. 4 is flow chart of steps of an embodiment of a method for proposing a synthesis pathway;

FIG. 5 is flow chart of steps of an embodiment of a method for proposing a synthesis pathway;

FIG. 6 is a diagram illustrating steps of an embodiment of a method for extracting a reaction template;

FIG. 7 is flow chart of steps in an embodiment of a method for proposing a reaction;

FIG. 8 is a flowchart of steps in an embodiment of a method for filtering out possibly incorrect reactions;

FIG. 9 is a flowchart of steps in an embodiment of a method for creating negative reactions;

FIG. 10 is a diagram illustrating an embodiment of a method for representing a reaction;

FIG. 11 is a flowchart of steps in an embodiment of a method for training a model for proposing a synthesis pathway;

FIG. 12 is a screenshot from an embodiment of a user interface displaying an embodiment of a pathway view;

FIG. 13 is a screenshot from an embodiment of a user interface displaying a detailed view of a reaction from a synthesis pathway;

FIG. 14 is a screenshot from an embodiment of a user interface displaying a target compound input screen;

FIG. 15 is a screenshot from an embodiment of a user interface displaying an embodiment of a screen in which a user inputs search parameters;

FIG. 16 is a screenshot from an embodiment of a user interface displaying an embodiment of a screen displayed while results are being generated;

FIG. 17 is a screenshot from an embodiment of a user interface displaying an embodiment of a screen displaying detailed views of partial search results;

FIG. 18 is a screenshot from an embodiment of a user interface displaying an embodiment of a screen displaying detailed views of partial search results;

FIG. 19 is a screenshot from an embodiment of a user interface displaying an embodiment of a screen displaying detailed views of finished search results;

FIG. 20 is a screenshot from an embodiment of a user interface displaying an embodiment of a screen displaying a full synthesis pathway for the results displayed in FIG. 19;

FIG. 21 is a screenshot from an embodiment of a user interface displaying an embodiment of a screen displaying reactions similar to the reaction of FIG. 19 and FIG. 20;

FIG. 22 is an example of a proposed synthesis pathway generated by an embodiment;

FIG. 23 is an example of an alternate compound;

FIG. 24 is an example of a proposed synthesis pathway generated by an embodiment using an alternate compound;

FIG. 25 is a screenshot from an embodiment of a user interface displaying an embodiment of a screen displaying grouped reactions;

FIG. 26 is a drawing showing an embodiment of a user interface displaying an embodiment of a screen illustrating supporting information;

FIG. 27 is an illustration of an embodiment of positive and negative reactions;

FIG. 28 is an illustration of an embodiment of a method for generating positive and negative reactions;

FIG. 29 is a chart showing correlation between an embodiment of a synthetic accessibility score and known scoring methods;

FIG. 30A, FIG. 30B, and FIG. 30C are charts showing comparative results from an embodiment of a synthetic accessibility score against known scoring methods for pathways with different numbers of reactions;

FIG. 31 is a flow chart showing an architecture for an embodiment of a method for proposing a synthesis pathway;

FIG. 32 is a drawing showing an embodiment of a user interface displaying an embodiment of a search tree of a method for proposing a synthesis pathway;

FIG. 33 is a drawing showing an embodiment of a user interface displaying an embodiment of a search tree and features of the search tree;

FIG. 34 is a drawing showing an embodiment of a user interface displaying an embodiment of a search tree and features of the search tree;

FIG. 35 is a drawing showing an embodiment of a user interface displaying an embodiment of a search tree and features of the search tree;

FIG. 36 is a is a drawing showing an embodiment of a user interface displaying an embodiment of a search tree and features of the search tree;

FIG. 37 is an illustration of an aspect of an embodiment of the method for proposing a synthesis pathway;

FIG. 38 is an illustration of an aspect of an embodiment of the method for proposing a synthesis pathway;

FIG. 39 is an exemplary block diagram depicting an embodiment of a system for implementing embodiments of methods of the disclosure; and

FIG. 40 is an exemplary block diagram depicting a computing device.

### DETAILED DESCRIPTION

### An Overview of an Embodiment

In the embodiments of methods for proposing a synthesis pathway to a target molecule, the embodiments leverage artificial intelligence to design chemical syntheses within seconds, instead of hours or days. In the embodiments, some of the intermediate reactions within any synthesis pathway may be entirely novel—in the sense that the intermediate reaction is created by the method, rather than filtered from reactions within an accessible database.

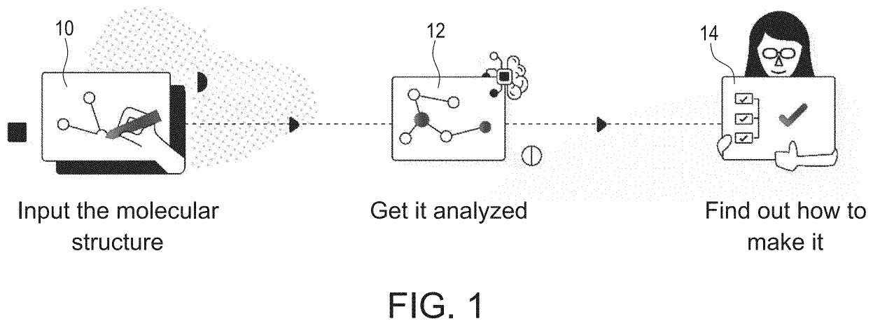

FIG. 1 illustrates an embodiment of a method for proposing a synthesis pathway. In a first step 10, a chemist (the prototypical user of the method) inputs the structure of the molecule that is the target of the synthesis, along with optional, additional criteria, into the system. In step 12, the user launches the system, which analyzes the target molecule and proposes synthesis pathways, as will be described in detail within. Generally, in step 12, the system determines pathways for synthesizing the target molecule from commercially available molecules. Finally, in step 14, the determined pathways are ranked, optionally according to user-defined criteria, and presented to the user. In embodiments, a proposed pathway may be accompanied with supporting, lab-tested evidence showing, e.g., reaction feasibility.

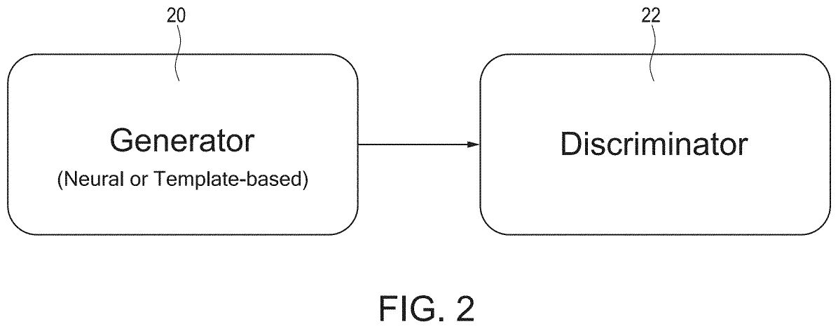

FIG. 2 illustrates an embodiment of elements of analysis step 12. In FIG. 2, the analysis applied a generator 20 to generate proposed synthesis pathways. Generator 20 may be template-based or neural-network based. After the generation of the proposed pathways, a discriminator 22 determines the probability or feasibility of the generated reactions.

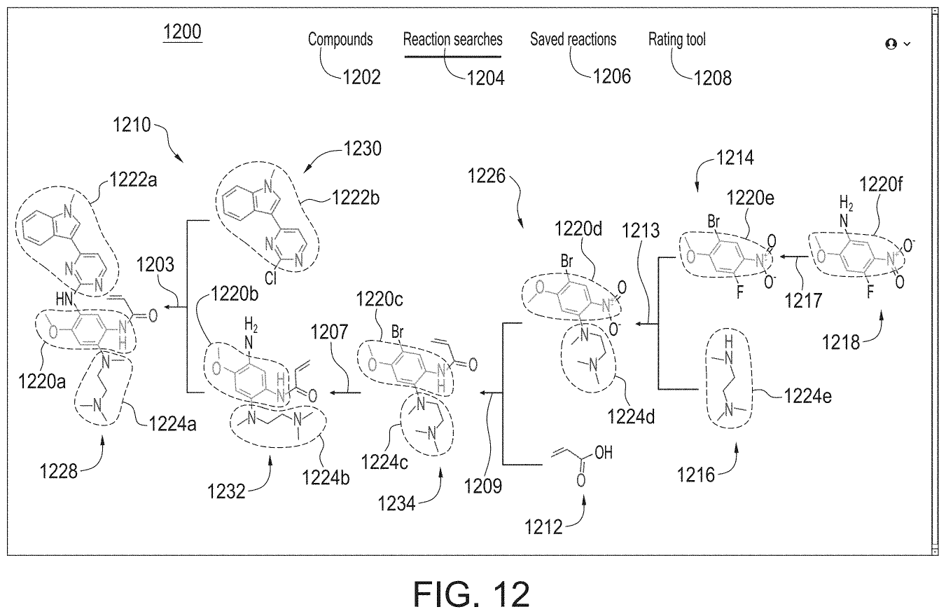

In an exemplary use of an embodiment, a user may enter a target molecule. For example, the structure of Osimertinib. The user may then choose synthesis criteria that are suitable for late stage drug discovery: medium quantity, short shipping time of starting materials. The system may then be launched. While first results may be available within seconds, complete results may require minutes of computation. In embodiments, the system employs deep learning—utilizing information about previous experiments to find out what kinds of transformations between different molecules are viable. The system is then able to propose novel synthesis steps that lead to previously unseen molecules. These synthesis steps are then assembled into a search tree that includes all proposed reactions from substrate to target molecule. From the search tree, pathways from starting materials to product are extracted and ranked. The pathway ranking may consider and account for the user-chosen criteria, which reflect real customer scenarios. With the search finished, the most promising result is shown to the user in a GUI (e.g., FIG. 12). On the left side of the screen the user sees their target molecule. With the aid of different colors, the user can trace individual atoms, or structural sections of the target molecule back to commercially available molecules. Thus, using embodiments, a process that previously required hours of chemist time—a process that needs to be iterated thousands of times to develop just one drug—may be performed in minutes.

### A Top-Level Description of the System Functionality

In embodiments, systems and software design organic synthesis pathways for desired organic molecules where the user inputs one or more structures of the molecule(s) they want to make.

In an embodiment, a pathway consists of a collection of starting materials (substrates) and one or more reactions leading from starting materials to the desired product (target molecule).

In an embodiment, the software utilizes multiple types of information, including databases of previously performed reactions (known or “referential” reactions), commercially available starting materials, and user-introduced parameters. In an embodiment, the software may allow the user to input this information into the system; however, the input of this information is not necessary for the system to function, as the absolutely necessary data are supplied with the system.

In an embodiment, the software may propose novel chemical reactions. These “novel” reactions, therefore, are not introduced into the system. Instead, they are generated “on-the-fly” when by the software. The system has a module for reaction feasibility estimation, which is discussed within. Regarding “novel,” as used above it means: created by the system and not retrieved by the system from a database. Thus the novel reaction may be different from any reaction that is within a database accessed by the system or otherwise supplied to the system. In other words, the novel reactions are not programmed into the dataset, but algorithmically generated. In simplest terms, rules of “what kinds of reactions are possible” are extracted from the reaction database, and then they are applied to any chemical compound, even unseen ones. This will be described later, in “reaction proposing” section. Thus, known reactions may be incorporated in the results, but a feature of an embodiment is the ability to generate reactions de novo.

In an embodiment, the software assembles the proposed reactions into multi-reaction synthetic pathways and ranks these pathways. This is discussed further regarding the Search Tree. Reactions are first assembled into the search tree structure and then pathways are extracted from that structure. In brief, the search tree includes all the different reactions that may be used to synthesize the target molecule. These reactions are included as, e.g., different offshoots, trunks, limbs, branches, or leaves, of the search tree. In an embodiment, compounds may be represented by compound nodes and reactions by reaction nodes. In the embodiment, to indicate a reaction, directional links may join compound nodes to a reaction node, and a directional links may join the reaction node to a product compound or nodes. In the embodiment, a single compound node may be both a product of one or more “upstream” reactions and a substrate for a single “downstream” reaction, where “upstream” and “downstream” are determined by the directional links. In the embodiment, a single compound may be linked to both multiple downstream reactions and multiple upstream reactions. That is, embodiments of the reaction proposing method may determine a plurality of ways to synthesize a particular compound (which may be, e.g., the user's target compound, or a substrate in a reaction proposed to synthesize the user's target compound). The reaction proposing mechanism may also determine several ways to employ that same compound as a substrate in a subsequent reaction. Thus, an embodiment of a search tree is an interconnected group of reactions leading from substrates to the user's target molecule.

In an embodiment, the reaction proposing mechanism may also propose an alternate, ultimate target molecule to the user that results from a synthesized substrate in a search tree with a commercially available substrate that is slightly different from the synthesized substrate. In this embodiment, the downstream reactions from the changed substrate are revised to reflect the change and the revised reactions become different branches of the search tree that lead to the alternate, ultimate target molecule. The user may then decide whether to have the alternate target molecule synthesized, either in addition to the user's original target molecule, or instead of the original target molecule.

In an embodiment, the ranking is done by multiple methods, including statistical and heuristics. The ranking is meant to represent the total estimated cost of pathway execution, including the cost of starting materials and the risk of synthesis failure. User preferences are considered and accounted for. For example, while total estimated cost may be the ultimate criteria, the total estimated cost may depend on user preferences, as described below regarding the cost function.

In an embodiment, the software provides a detailed view of each reaction and compound, including supporting information, such as reaction execution conditions, prices, and availability of starting materials, based on information within the system and information introduced by user. The supporting information serves also as the basis for the system's decision, which in this context includes: the entirety of the system's reasoning: what reactions to propose, what is their feasibility, how their cost is estimated, what synthesis paths get shown to the user etc.

In an embodiment, a GUI allows the user to view the proposed pathways and interact with them. The user may have a large influence on the direction in which the planning process goes. For example, using the GUI, the user may pick the compound in the search results that should be analyzed more thoroughly, and the user may also change the behavior of the search policy, as described below.

In an embodiment, the user may export the search results and all information provided by the system in different formats. They may also save the queries and the search results for later use.

In an embodiment, the input and constraints that the user may introduce may have a profound effect on the reactions proposed. For example, user input constraints may include: the amount of the target compound that is desired, restrictions on the availability of equipment and reagents (including, e.g., constraints based on the supply chain for each substrate), constraints regarding the categories of reactions that may be used in the synthesis pathway, and constraints regarding details of the target molecule (e.g., bonds in the target molecule that may not be broken during the synthesis pathway). Typical software simply allows parameters to be specified that are much less relevant to the use-case, such as maximum number of reactions in the synthesis plan, maximum price per quantity of starting materials, scoring function type A or B, etc.

There are two primary use cases. In a first use case, the user defines what end-products to synthesize. In a second use case, the system generates a library of similar compounds based on user-defined constraints and proposes synthesis pathways for each compound in the library. In the second use case, it may be much cheaper to synthesize multiple similar compounds at once than synthesizing each of the compounds separately. This is because one can reuse intermediate compounds and starting materials that are common for synthesis plans of each end-product (sort of “economies of scale”). For the second use case and the generation of a library of similar compounds (e.g., based on user constraints, or based on a similarity to a user-selected target end-product), the system may propose a reaction pathway for one similar compound that has no intermediate or starting substrate in common with a reaction pathway proposed for a different similar compound, or in common with a user-proposed target compound.

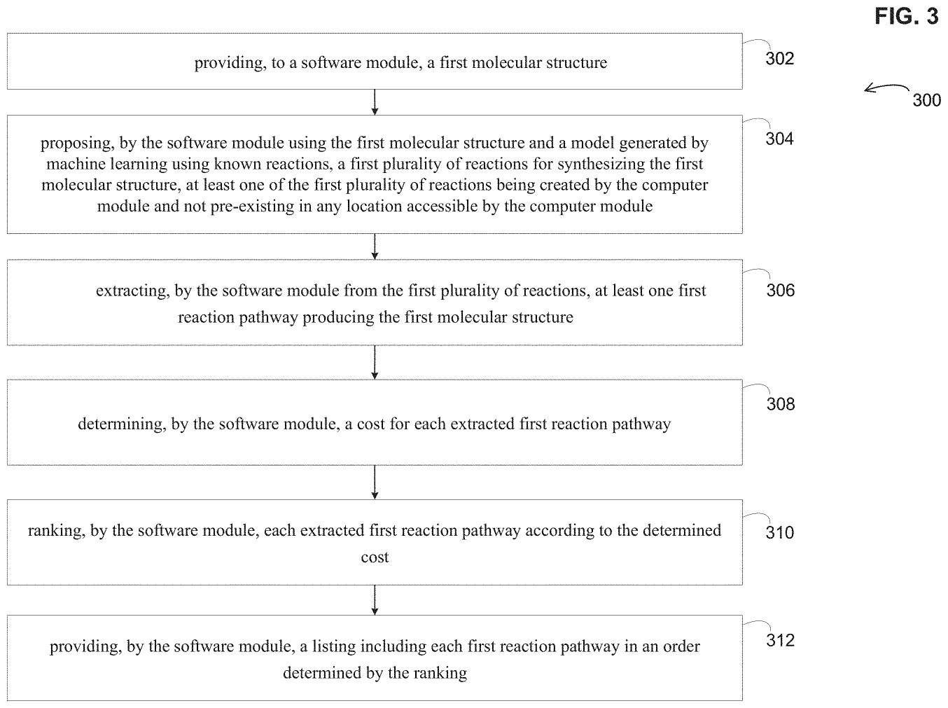

FIG. 3 is a flow chart of an embodiment of a method 300 for proposing a synthesis pathway. In step 302, a first molecular structure is provided to a software module. The molecular structure would typically be provided by a user through a GUI. In step 304, the software module would propose a first plurality of reactions for synthesizing the first molecular structure, where at least one of the first plurality of reactions being created by the computer module is not pre-existing in any location accessible by the computer module. In this proposing step, the software module would use the first molecular structure and a model generated by machine learning using known reactions. In step 306, the software module would extract, from the first plurality of reactions, at least one first reaction pathway producing the first molecular structure. In step 308, the software module would determine a cost for each extracted first reaction pathway. In step 310, the software module would rank each extracted first reaction pathway according to the determined cost. And in step 312, the software module would provide a listing including each first reaction pathway in an order determined by the ranking.

Aiding Retrosynthesis with Statistical Models

In embodiments, a primary feature of the software is the ability to propose chemical reactions leading to a target compound. This is done with the help of machine learning models, which use information about previously carried out successful reactions, referred to within as positive or “referential reactions.” In embodiments, models may also be trained using both positive reactions and negative reactions, where the negative reactions include information about known unsuccessful reactions, or information about proposed reactions that are designated to be “infeasible,” or both known unsuccessful reactions and proposed, infeasible reactions.

### Proposing Candidate Reactions for a Target Compound

In a typical method of retrosynthesis, in response to the input of a chemical compound by a user, the system outputs a number of candidate reactions leading to the molecule. The number of candidate reactions may be extremely large, and so, in embodiments, the number may be limited. In the typical method of retrosynthesis, this is done by a reaction generator, which may use any one of several techniques. 1) Reactions may be generated by applying templates to the target compound. Reaction templates for single step retrosynthesis are rules to rewrite the target into substrates. In the context of synthesis planning software, reaction templates are usually automatically extracted from reaction data. They can be also manually curated and include a set of conditions under which a template can be applied. A statistical model may be trained on the dataset of referential reactions. It may be realized in many ways. One example is a pair of neural networks, where the first network predicts a place in the target compound where the reaction occurs, and the second network generates a full reaction based on the target and the reaction place. 2) The system may search for referential reactions, where the product is similar to the target compound. To measure similarity between compounds well established techniques may be used, such as molecular fingerprints. In an embodiment, some number of the most similar referential reactions are used where the reaction place matches the target compound and apply them to acquire candidate reactions.

Individually, the former approaches may be known methods for retrosynthesis. However, in an embodiment, our system may combine these approaches in a novel way. The statistical model may be used to aid search in the database of referential reactions. These methods may benefit in both directions: relevant referential reactions can reinforce the statistical models, and statistical models can improve searching in referential database.

The statistical model may be trained so that the search is most effective on the dataset of referential reaction, i.e. for a product from a referential reaction, the corresponding referential reaction is proposed as often as possible. This may be done in any of several ways. 1) Training a model that learns a similarity function between compounds. This may be used to make the similarity measure more relevant to the retrosynthesis task. 2) Training a model that predicts some properties of desired referential reactions (e.g. type of reaction). Referential reactions may then be limited to only those which match some predicted criteria and are probably more relevant for the user.

### Input Interface Description

In an embodiment, the input interface is a tool that allows to input the structure or structures of desired molecules via one or more of: machine-readable formats like SMILES, chemical table file; a plugged-in external molecular editor; searching the structure in an external data source that has been integrated with the software; Automatically via API; or a built-in molecular editor.

In an embodiment, the input interface is a tool that allows the user to introduce data and preferences used in the pathway design process. For example, the interface may be used to: plug in external data sources; and/or to introduce information directly through the interface, concerning starting materials, ranking preferences, reaction conditions and other factors influencing the search.

### Search Tree

In an embodiment, a search tree is a basic data structure which the system may use to assemble synthesis pathways.

In an embodiment, a search tree may be a directed graph composed of reaction nodes and chemical compound nodes. At the beginning of the search, the search tree may consist of a single chemical compound node—the root of the tree that represents the product. The structure of the tree is a direct result of iterations (“expansions”) described below.

A search tree is structurally similar to a synthesis pathway. The main difference between a synthesis pathway and a search tree is that in a search tree there may be multiple reactions that yield a given chemical compound. Conceptually, a search tree represents the set of all possible synthesis pathways that may be assembled from reactions that we proposed during the search.

In an embodiment, a pathway assembling algorithm works by iteratively “expanding” the search tree, and then extracting synthesis pathways from it. Extracting synthesis pathways may be done after any number of iterations, thus it allows the system to show partial results of the search to the user even before the search finishes.

In an embodiment, extracting all synthesis pathways and/or several of the best synthesis pathways and/or a subset of pathways that comply to certain constraints/ . . . from search tree may be done using standard dynamic programming approaches.

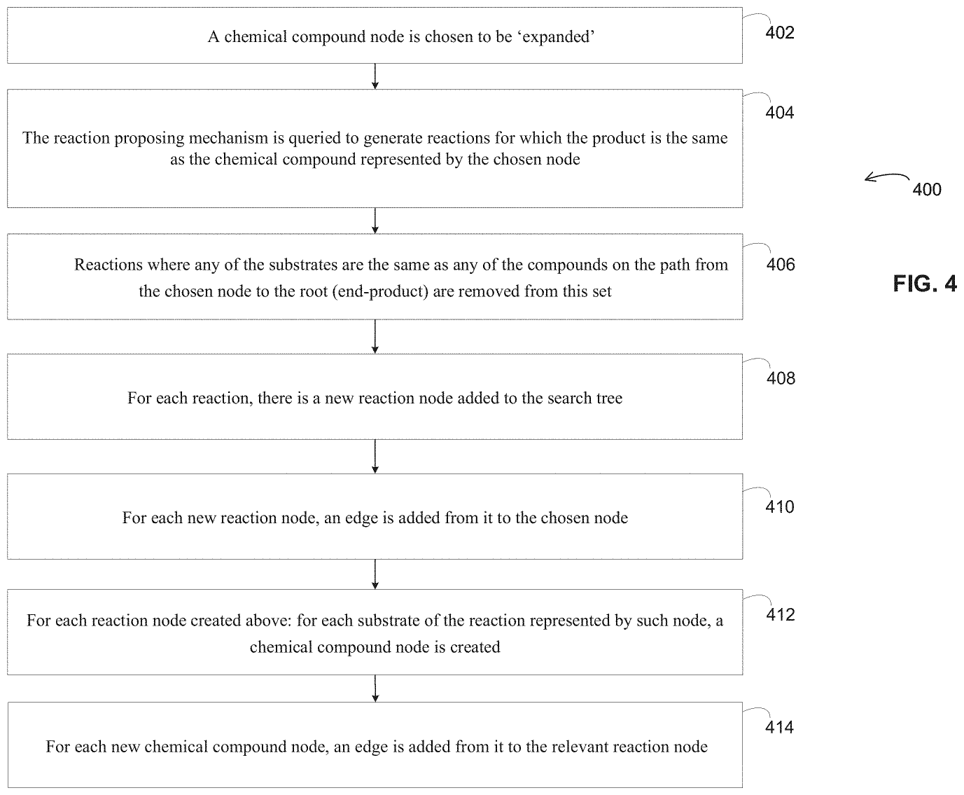

FIG. 4 is flow chart illustrating steps of an embodiment of a method 400 for expanding a node of a search tree. Method 400 describes an iteration of an expansion of a node of a search tree, where many such iterations may be performed on any search tree. In step 402, a chemical compound node is chosen to be “expanded.” It may be chosen according to the search policy or to user actions. For example, if the user requests that the system devote more time to analyze a certain compound, the search policy may be constrained by that request. In response it will choose a node from the subset of nodes that belong to the subtree of the node that represents the compound of user's choice. Usually in such case, there will be multiple iterations executed with such constraints. In step 404, the reaction proposing mechanism is queried to generate reactions for which the product is the same as the chemical compound represented by the chosen node. In step 406, reactions where any of the substrates are the same as any of the compounds on the path from the chosen node to the root (end-product) are removed from this set. This removal is done to avoid syntheses that contain loops (“make A from B, then make B from A”). In step 408, for each reaction, there is a new reaction node added to the search tree. In step 410, for each new reaction node, an edge is added from it to the chosen node. In step 412, for each reaction node created above: for each substrate of the reaction represented by such node, a chemical compound node is created. And in step 414, for each new chemical compound node, an edge is added from it to the relevant reaction node.

As a result of the process, each chemical compound and each chemical reaction may be represented as a node multiple times in the search tree. Each of those nodes has a different path from it to the root, representing different ways of utilizing a given reaction or compound in the synthesis process.

For each node in the search tree, there may be additional data and/or statistics stored in memory and updated upon each expansion to improve the performance of the algorithm or allow the function of the search policy/scoring algorithm.

### Cost Functions and the Estimate of Total Estimated Cost of Synthesis Pathway

In an embodiment, cost functions are used for calculating a total estimated cost of a synthesis pathway and for the purpose of search policy. There are multiple variants of cost function. An exemplary cost function used for calculating total estimated cost of a synthesis pathway is described as follows.

A cost function is calculated for each reaction node and compound node in the synthesis pathway. The value of the cost function of the end-product is the total estimated cost of synthesis pathway.

A cost function for a compound node that is a starting material (a leaf in the search tree) equals the price of the compound represented by compound node. It depends on many of the search parameters. For example: If a user requests that each starting material be available from multiple vendors (it is useful because vendors may be unreliable), the algorithm picks the price from the n-th cheapest vendor for given chemical compound (where n=number of vendors that user wants the starting materials to be available) instead of the cheapest one. In general, there may be many ways to incorporate the requirement for the redundancy of vendors of starting materials into the calculation price of starting material. The price for the starting material may be influenced by the amount that is required for the synthesis. This amount is calculated based on the amount of the final product that user wants to synthesize passed in the parameters, and on estimated yields and stoichiometric excesses of each reaction on the path from the starting material node to the end product. (Each reaction incurs some loss because of non-100% yield and thus requires usage of a larger amount of the substrate). A user may disallow or make preferred vendors (In an embodiment, a user may pick vendors from a list in the search parameters screen). Offers for compounds with shipping times greater than times requested by the user may be discarded, or the estimated time of shipping of starting materials may be incorporated into the price of starting material by putting a price tag on each day of the delay (the database of available compounds contains estimates of shipping times). The second approach allows the embodiment to account for the fact that long shipping times may be acceptable if the synthesis path itself is short. The embodiment may utilize a user-supplied database of chemical compounds available for the user or user's procurement data.

Other compound nodes in the synthesis pathways may be products of some reaction in the synthesis pathway. Cost function for each of these compound nodes equals cost function of the corresponding reaction.

A cost function of the reaction node is an estimated cost of executing a given reaction, including the cost of the substrates, the cost of chemists' labor etc. In an embodiment, the cost function=(sum of cost functions for each substrate node+linear factor*amounts of substrates+constant factor)*1/probability of success.

The probability of success may be derived using the reaction feasibility prediction model, described in other sections. The (1/probability of success) factor allows the embodiment to account for the fact that, in the case of failure, the compound has to be created again, probably in a completely different way.

The linear factor may represent the cost of executing chemical reaction that grows approximately linearly with the amounts of substrates that need to be taken into a reaction, which includes the cost of catalysts, the cost of solvents, etc. In an embodiment, a simplest implementation assumes the same value of linear factor for every proposed reaction. Its value can be approximated by considering average prices of solvents and catalysts used in chemical synthesis (for example, a very common solvent is THF that costs 100$/Liter, and usually for every mole of the substrate a reaction will need 1 L of the solvent, etc.). Having more precise data about reactions executed in the past, an embodiment will be able to look up the most appropriate solvent, and catalysts and conditions for the proposed reaction, and estimate that value in a more precise way.

The constant factor represents the cost of a chemist's labor required to actually execute chemical synthesis, and its value may be directly or indirectly derived from the search parameters (A user may input the cost directly or the embodiment may assume some constant value, as was done for the linear factor).

The amounts of the substrates are calculated based on the amount of the final product that the user wishes to synthesize, as described before.

One of the examples of how parameters influence which pathway is presented to the user is when small amounts of end product are requested. In that case, the cost of executing reactions (constant factor) dominates the cost of starting materials and causes shorter paths to be presented to the user as the best ones, even if the starting materials are relatively expensive. Conversely, for large amounts of the final product, it is more economically reasonable to use small, very cheap starting materials even if more reactions need to be executed. This behavior (large amount leads to long syntheses, small amount leads to short syntheses) matches the users' expectations, and is an emergent behavior, i.e., a behavior not encoded explicitly in the system.

Thus, in embodiments, the calculation of the cost of an extracted pathway is directed to providing an actual cost of executing the pathway synthesis, rather than an abstract measure of synthesis complexity.

### Search Policy (Algorithms Governing the Design Policy)

In an embodiment, the search policy is responsible for picking nodes that will be expanded during the search. In an embodiment, the search policy may utilize a variant of the cost function—“search policy cost function”—described below. For each unexpanded node in the search tree, the cost of the cheapest (in terms of search policy cost function) synthesis pathway that contains given node is calculated—the lower this cost is, the better. Then, one or several best nodes are chosen to be expanded. For the purpose of the search policy, those synthesis pathways do not need to have starting materials that are commercially available.

In an embodiment, if the user wants some compound to be analyzed more thoroughly, the embodiment limits the set of nodes chosen from the search tree to those nodes that belong to the subtree of the node representing given compound.

In an embodiment, the main difference between search policy cost function and cost function described before is that, for the purpose of search policy, the embodiment does not use the price of the starting material, but rather it's estimation, described below. The mice estimation serves the same purpose as an evaluation function in the A* algorithm (which is an algorithm known by those of skill for use in finding the shortest routes in graphs) and the whole search algorithm may be considered a heavily modified variant of the A* algorithm, where we look for the cheapest subtrees (i.e., the cheapest synthesis pathways) of the search tree instead of searching for the shortest routes in a graph.

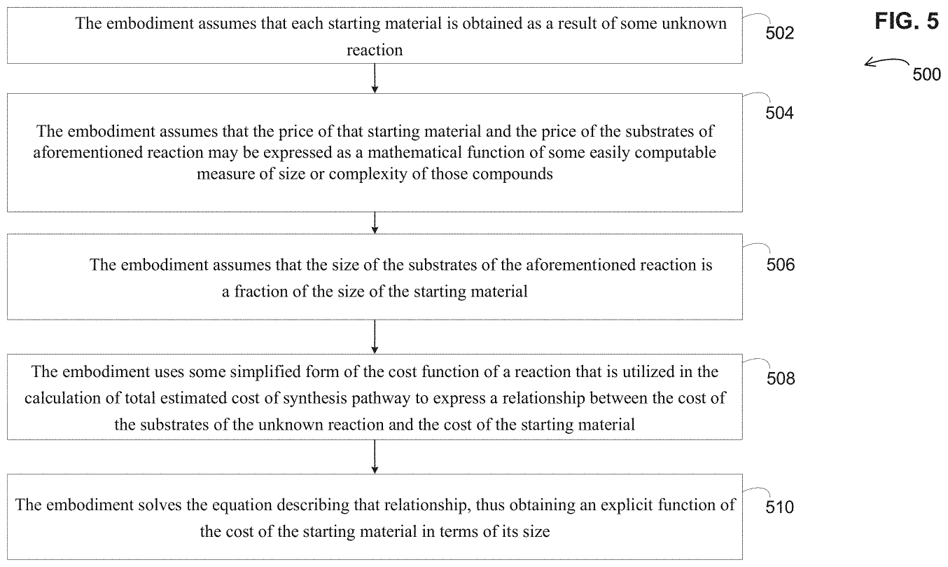

FIG. 5 is a flow chart illustrating steps in an embodiment of a method 500 for cost estimation. In step 502, the embodiment assumes that each starting material is obtained from some unknown reaction. In step 504, the embodiment assumes that the price of that starting material and the price of the substrates of the unknown reaction may be expressed as a mathematical function of some easily computable measure of size or complexity of those compounds (for example, a number of non-hydrogen atoms). In step 506, the embodiment assumes that the size(s) of the substrates of the unknown reaction is a fraction of the size of the starting material. In step 508, the embodiment uses some simplified form of the cost function of a reaction that is utilized in the calculation of total estimated cost of synthesis pathway to express a relationship between the cost of the substrates of the unknown reaction and the cost of the starting material. In step 510, the embodiment solves the equation describing that relationship, thus obtaining an explicit function of the cost of the starting material in terms of its size. In an embodiment, it is assumed that: (1) the cost of starting material, or the substrates of the unknown reaction, is proportional to the amount of that compound, (2) the reaction requires two substrates of the same size, and (3) the constant factor of the cost of the reaction is negligible. Thus, the embodiment arrives at the equation:

f(x)=(r+f(kx)·2/y)·1/p Equation 1

### Where:

x=size of the starting material,

f(x)=price of the starting material

k=substrate to product size ratio,

kx=size of the substrate of unknown reaction

y=yield of the unknown reaction

r=linear factor of the reaction cost

p=probability of success of the unknown reaction.

By specifying the boundary condition: f(x₀)=f₀, the embodiment can solve Equation 1 above and obtain:

f(x)=(q+f₀)(x/x₀)^{ln(y·p/2)/ln(}k)−q Equation 2

where q=r·y/(2−p·y). This equation may be used directly by the system to calculate an estimated price from the size of the starting material. Thus, the embodiment may calculate the cost of synthesis pathway even when the starting materials are not available.

In an embodiment, the values of the constants r, p, y, k are chosen, if it is possible, to match the constants in the cost function used for the calculation of total estimated cost of the synthesis pathway.

An example of a case when it is not possible is the probability of success, as it is calculated on a per-reaction basis using machine learning models. Thus, for the purpose of price estimation, in an embodiment some optimistic value is manually chosen based on the distribution of the probabilities that the model outputs. That ensures that the price estimates are optimistic, and that gives the algorithm a high chance of finding an optimal solution—just like an admissible heuristic (i.e. one that does not overestimate the cost of the goal) in A* algorithm ensures that an optimal route is found.

In an embodiment, the boundary condition values (x₀, t₀) are currently chosen manually to match the average size of the starting material used commonly in organic synthesis, and the cost of the starting material that is considered reasonable by most chemists.

In an embodiment, one improvement is a more fine-tuned size calculation: instead of calculating a number of non-hydrogen atoms, a weight is assigned to each non-hydrogen atom in the molecule. These weights are summed to yield the size of the molecule for the purpose of estimating price. Weights may be calculated in the following manner. First, a set of graphs is generated offline (before the start of the search), and a factor assigned to each of graph. To calculate the weight of an atom in a compound during the search, the system finds all subgraphs from the set of graphs that contain the atom of interest. The weight is a product of all the factors that are assigned to those graphs.

In an embodiment, manually picking subgraphs and their factors is done by considering frequently occurring fragments of the molecules that are making the synthesis of the molecule harder (where a factor greater than 1 is assigned), or easier (where a factor lower than 1 is assigned). This process may be automated by algorithmically finding the set of most frequently occurring subgraphs in the molecules available in the dataset of commercially available compounds, and then assigning the factors of those subgraphs by means of statistical regression so that estimated prices calculated using sizes based on those factors match the actual prices that the system has access to via the database of commercially available compounds. In the same way, constants of the equation for estimated price may be fitted.

In an embodiment, the search policy described above may be mixed with other approaches by parallel selection of expansion nodes using this search policy and other policies (random or weighted random, BFS, search policy with different—more or less optimistic—sets of parameters, etc.), and using techniques such as running iterative deepening starting on the node selected by a search policy etc.

### Reaction Proposing

In an embodiment, a reaction proposing method is based on a set of templates generated from a database of previously executed reactions.

In an embodiment, each template may be algorithmically generated from a reaction. A template encodes information about: 1) the changes in a graph structure of the substrates that occur as a result of the reaction, and 2) a neighborhood of the atoms that belonged to the parts of the graph that were changed.

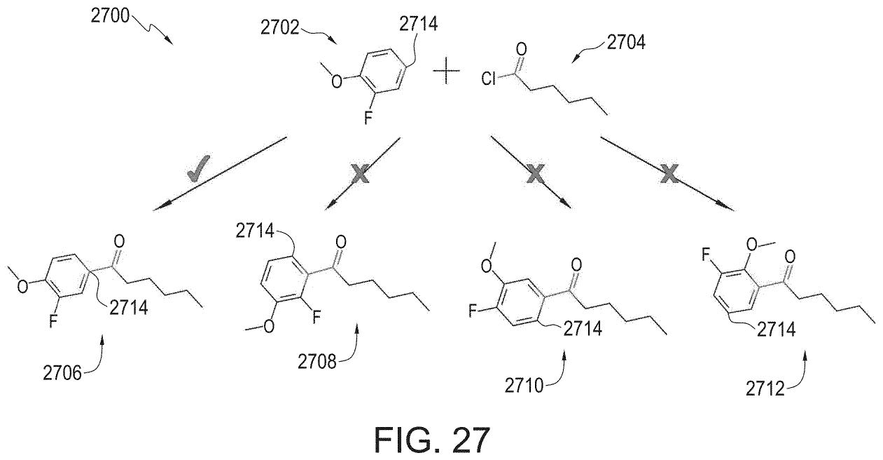

In an embodiment, multiple reactions may yield the same template. For example, all reactions in FIG. 27 yield the same template. In the case of datasets that may contain errors, the number of reactions in the dataset that yield a particular template is used as a rough method for filtering out reactions, as erroneous reactions tend to yield templates that are very infrequent.

In an embodiment, template generation algorithm requires input in the form of: 1) a graph of substrates, 2) a graph of a product, and 3) information about mapping, that is, information about what atom in the product corresponds to what atom in one of the substrates.

In an embodiment, a template generating algorithm does not require substrates or products to be fully mapped (that is, not every atom in substrates needs to have a corresponding product atom and vice versa) and the algorithm is designed to fix inconsistencies in the mapping.

In an embodiment, the elements in the substrates and the product do not have to be balanced (that is, they do not follow this quotation from Wikipedia: “The law of conservation of mass dictates that the quantity of each element does not change in a chemical reaction. Thus, each side of the chemical equation must represent the same quantity of any particular element”), so the algorithm tolerates reactions where some of the substrates are omitted (for example, in the case of ester hydrolysis it is obvious that water molecule needs to be included in some form in the substrates of reaction equation), or where side products are omitted.

In an embodiment, mapping information may not be duplicated, that is, there should be no substrate atom that has more than one corresponding product atom or vice versa. Note: Such duplicated mapping may sometimes be generated by certain mapping algorithms to note the fact that some substrate is used “more than once” in the reaction—stoichiometry different than 1:1 where multiple molecules A react with one molecule B.

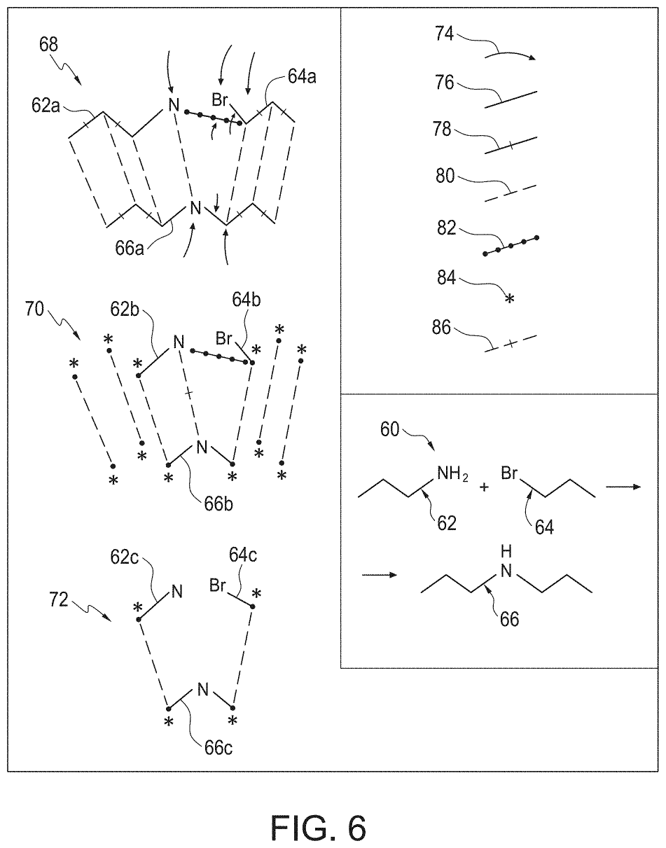

FIG. 6 is a diagram illustrating an embodiment of a method for constructing a reaction template 72. In FIG. 6, atoms or bonds that have changed are indicated by arrows 74. Single bonds are indicated by lines 76. “Boring” bonds that are removed are indicated by cross-hatched lines 78. Special mapping edges are indicated by dashed lines 80. Special “missing bond” edges are indicated by dotted lines 82. Wildcards are indicated by asterisks 84. And mapping edges between non-wildcard atoms that were removed are indicated by cross-hatched, dashed lines 86. In FIG. 6, from a reaction 60 between substrates 62, 64 that react to create a product 66, an initial graph 68 is the sum of subgraphs, i.e., substrate subgraphs 62, 64 and a product subgraph 66.

In an embodiment, and with reference to FIG. 6, a template construction method may be conceptually separated into phases: 1) Annotation: For both the substrates 62, 64 and product 66, for each atom and each bond, the embodiment may determine their features (e.g., information whether a given atom or bond is part of some ring or a ring of certain size, whether the atom or bond belongs to some certain subgraph, etc.) and annotate bonds and atoms with the features, e.g., one or more of indicators 74 . . . 86. Each atom may be additionally tagged with information regarding whether it is a part of one of the substrates or the product. 2) Merger: the embodiment may create a graph 68 that is a simple sum of substrate 62a, 64a and product 66a graphs (FIG. 6). Then, in graph 68, according to the mapping data, added as input, the embodiment of the template generation process may add a special “mapping” graph edge 80 for each pair of corresponding substrates 62a, 64a and product 66a atoms. Then, for each bond edge 76 between substrate atoms, where the bond is not in the substrate but is found in the product, the embodiment may add a special “missing bond” edge 82, e.g., between nitrogen of substrate 62a and the carbon of 64a. 3) Extraction of the reaction core: the embodiment may modify graph 68 to graph 70 by selecting the “boring” (explained below) bonds 78. Each atom connected by such a bond is marked as a wildcard 84, and the boring bonds 78 are removed. Graph 70 is modified to reaction template 72 by the following. Mapping edges 78 between non-wildcard atoms are removed. “Missing bond” edges are recalculated (they are removed and added again according to the same rules as before). A missing bond edge 84 is recalculated as follows: as the mapping edge is removed, the nitrogen atom in the product no longer has corresponding atom in the substrate and thus the bond is no longer considered missing. Connected components of the graph 70 that do not have any wildcard atoms are discarded (which is not applicable to graph 70). Thus, substrate 62b, 64b and product 66b are retained. Connected components of the graph that do not have any atom that has a corresponding atom and that has changed during the reaction are discarded. “Changed” means that its charge changed, or it is connected by a bond that changed during the reaction. Thus, the outer two special mapping edges 80 on each side of graph 70 are discarded. Mapping edges 86 are removed from the non-wildcard atoms (N) for the purpose of unifying different ways of mapping reactions of the same type, the benefit of which is explained with regard to Equation 3. In Equation 3, for the esterification reaction as drawn, there are six different ways of mapping oxygen atoms in the substrates onto oxygen atoms in the product, although distinction is irrelevant for the purpose of generating new esterification reactions. Thus, the template generation method as described above would result in a single template.

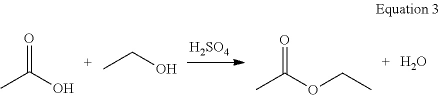

In an embodiment, “boring” edges are edges that are not interesting. All “mapping” and “missing bond” edges are interesting. All bond edges that: have no corresponding edge, or whose corresponding product bond edge is interesting, or whose corresponding bond is different (that is, the corresponding bond was modified during a reaction) are interesting.

Considering those bonds as interesting (and thus not removing them in the process of extracting a template) is necessary to encode changes in the graph structure of substrates that occur during the reaction.

In an embodiment, other edges are considered interesting so that qualitatively different reaction types will yield different templates, such as differentiating between: “ester formation from acyl halide and alcohol” or “Williamson ether synthesis.” This also helps with unifying different ways of mapping the reactions of the same type. Other bonds that may be considered interesting in embodiments include: 1) All double and triple bonds that are not part of an aromatic ring; 2) All bonds that do not connect a neutral carbon atom with a neutral carbon atom, and that are not part of aromatic ring, and 3) All bonds that do not connect a neutral carbon atom with a neutral carbon atom, that connect at least one changed atom (changed atoms are defined in “Extraction of the reaction core”).

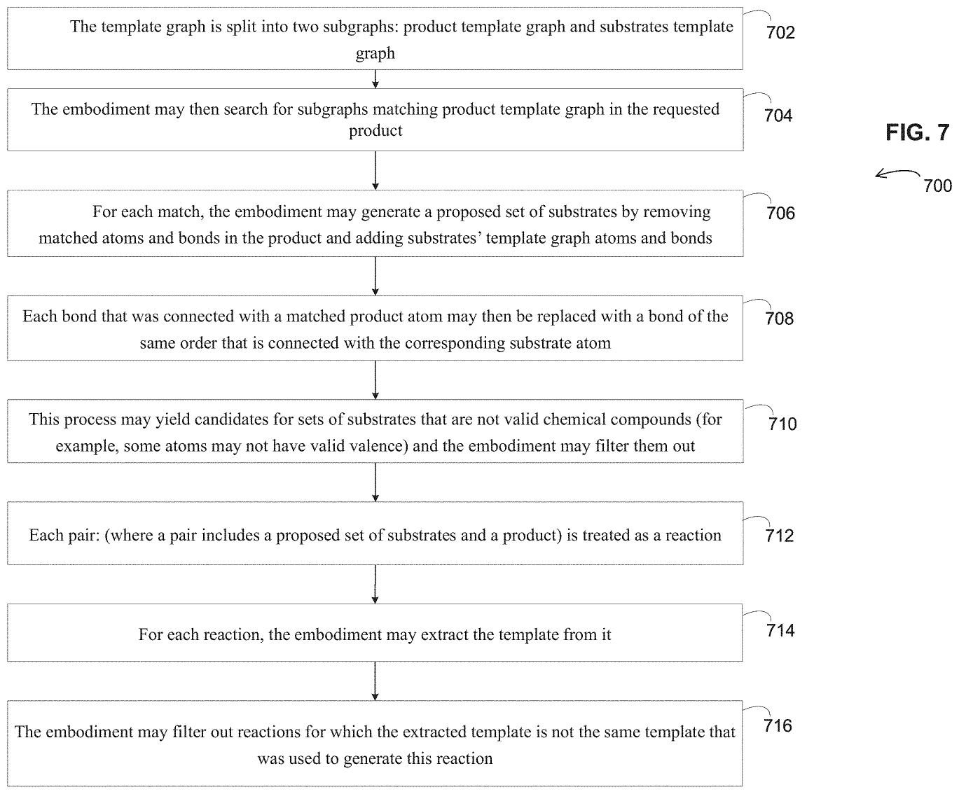

FIG. 7 is a flow chart illustrating steps of a method for proposing a synthesis pathway. In the embodiment, to propose reactions that yield the requested product, based on a particular template, the following method may be used. In step 702, the template graph is split into two subgraphs: product template graph and substrates template graph. In step 704, the embodiment may then search for subgraphs matching product template graph in the requested product. In step 706, for each match, the embodiment may generate a proposed set of substrates by removing matched atoms and bonds in the product and adding substrates' template graph atoms and bonds. In step 708, each bond that was connected to a matched product atom may then be replaced with a bond of the same order that is connected with the corresponding substrate atom. In step 710, this process may yield candidates for sets of substrates that are not valid chemical compounds (for example, some atoms may not have valid valence) and the embodiment may filter them out. In step 712, each pair: (where a pair includes a proposed set of substrates and a product) is treated as a reaction. In step 714, for each reaction, the embodiment may extract the template from it. In step 716, the embodiment may filter out reactions for which the extracted template is not the same template that was used to generate this reaction. This equality check is done based on checking the graph isomorphism and the annotations generated during template creation.

In an embodiment, this process may also be used to generate possible products based on requested substrates, by reversing the role of the substrate template graph and product template graph. Note: Representation of a reaction as a pair: (graph of set of substrates, graph of product) used in the description above is related to the representation of the reaction used by machine learning models by the facts that it does not require elements to be balanced nor the reaction to be fully mapped, but is otherwise dissimilar.

Regarding an embodiment of the reaction proposing method, a first plurality of reactions for synthesizing an exemplary target molecule of average complexity may result in the system performing computations for approximately three minutes and result in proposing, e.g., 17,000 reactions. From this set of reactions, the extracted pathways include those pathways that satisfy any user-supplied constraints, ranked in the order of lowest cost.

### Reaction Feasibility Estimation

In an embodiment, another feature of the system that uses machine learning is the reaction feasibility estimation. A reaction feasibility estimation may be provided directly to the user, and may be used as a method for ranking candidate reactions proposed in a retrosynthetic step. Similar to the proposing of candidate reactions, the embodiment may use the dataset of referential reactions to estimate the feasibility of a candidate reaction. 1) The embodiment may use a similarity measure (e. g. using reaction fingerprints) to find the most similar referential reaction to the candidate reaction and estimate the reaction feasibility as the reciprocal of the distance to the “nearest” referential reaction. Reaction fingerprints are known by those of skill and may be used to represent a reaction as a fixed length vector of bits. There are known metrics that may be used to measure distance between reactions (e.g., candidate reaction and referential reaction), such as Euclidean distance or Jaccard index. 2) The embodiment may estimate the reaction feasibility with statistical methods: Such methods involve building (learning) a statistical model (with machine learning, or more specifically, deep learning techniques) based on a dataset of chemical reactions. Referential reactions are the main source of data. In statistical models, the embodiment may use a custom reaction representation as an undirected graph, which is described below regarding the “chemical reaction representation.” The embodiment may treat the referential reactions as “positive” ones, i.e. reactions that occur in reality and generate “negative” (infeasible) reactions using custom heuristics. There are two versions of statistical models, described below, in Reaction Feasibility Estimation.

In an embodiment, regarding reaction feasibility estimation, two novelties may be introduced: 1) Constructing a statistical model able to discriminate chemical reactions generated by the system but deemed to be chemically improbable due to their low similarity to the referential reactions dataset. The main advantage of this approach is the construction of a dataset (that is used in training the model) with a significant part of the dataset consisting of reactions generated by our system, but considered infeasible. There are two versions of the model that are trained using different types of generated “negative” (infeasible) reactions, described below in “statistical models for reaction feasibility estimation.” Two methods of generating these negative reactions are described within the section on Statistical Models For Reaction Feasibility Estimation. In these methods, each reaction marked as “negative” is considered infeasible for the purpose of training the machine learning models. The reasoning that reactions generated by the system are, in fact, infeasible is heuristic, which may, in actuality, be incorrect in case of some of the “negative” reactions. 2) These statistical models use a custom reaction representation as an undirected multigraph with atoms represented as graph nodes and different kinds of edges representing chemical bonds in reaction substrates and product, discussed below regarding the “chemical reaction representation.”

### Statistical Models for Reaction Feasibility Estimation

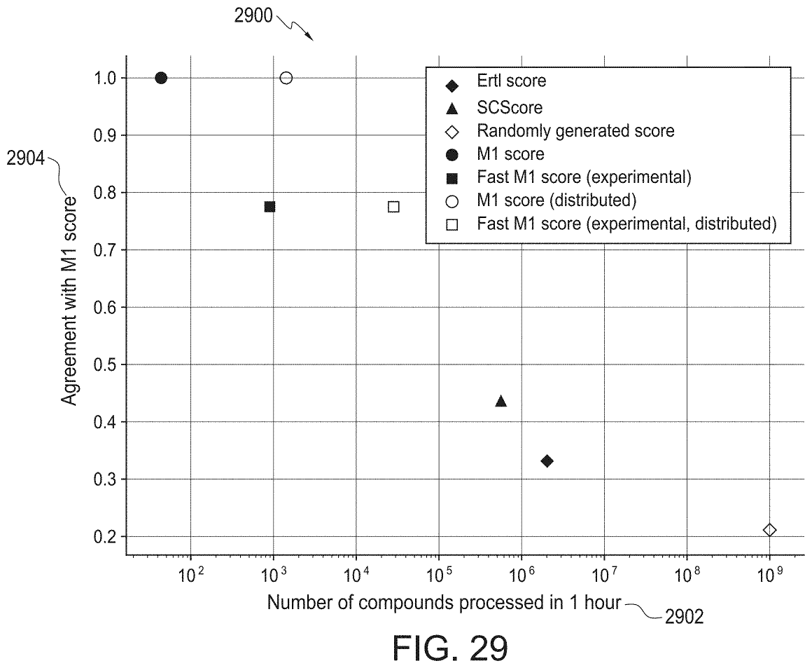

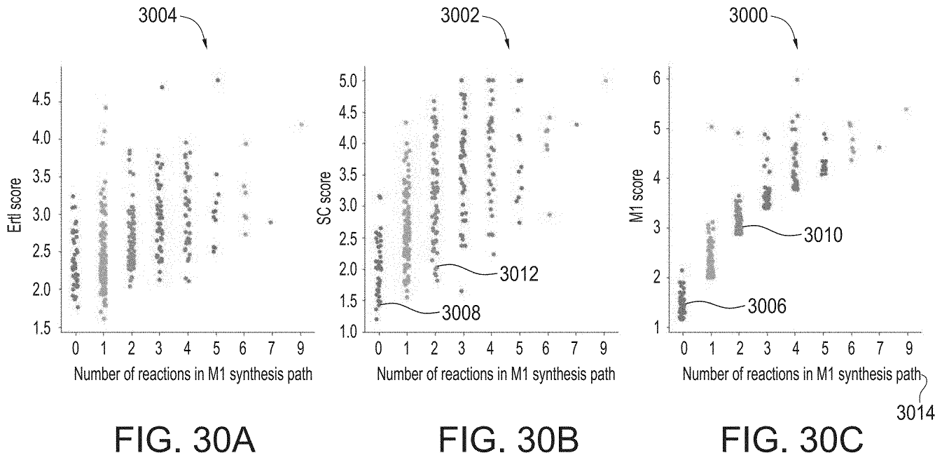

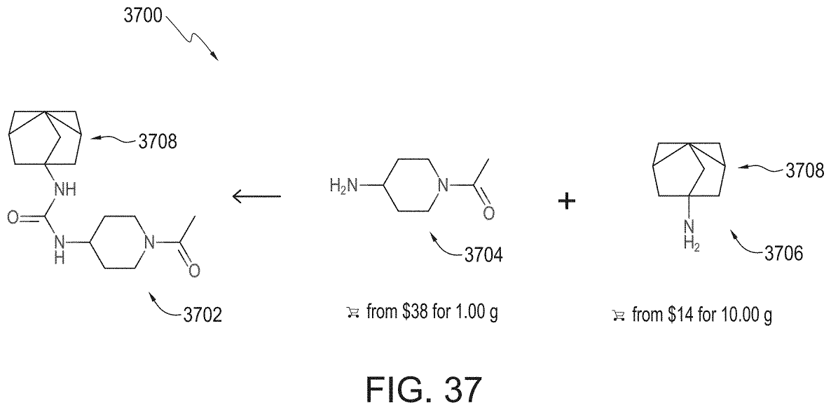

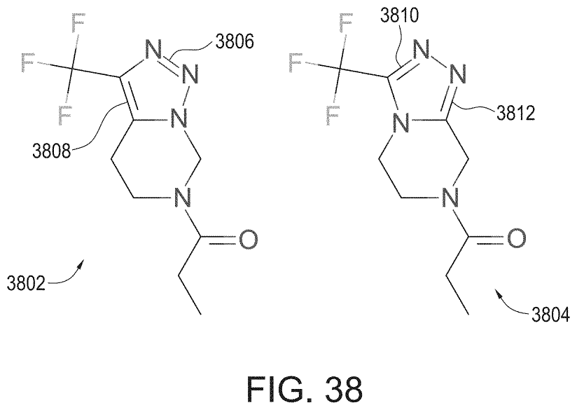

An embodiment may introduce two machine learning approaches for estimating reaction feasibility using the referential reactions dataset: the first models the probability that a given chemical reaction occurs; and the second discriminates chemical reactions generated by the system that do not match the distribution of data represented by referential reactions. In an embodiment, a measure of a reaction feasibility estimation developed according to the following discussion is called a synthetic accessibility score (SAS), which is also discussed further within with reference to FIG. 29, FIG. 30C, FIG. 37, and FIG. 38.

Based on experiments, using both approaches for training gives the most powerful statistical model for estimating reaction feasibility.

1. Modelling Probability that a Given Chemical Reaction Occurs

This type of model may be used to aid retrosynthesis by ranking reactions by their probability or filtering out improbable reactions. However, typical models are not adjusted specifically for, or simply do not address, the retrosynthetic setting.

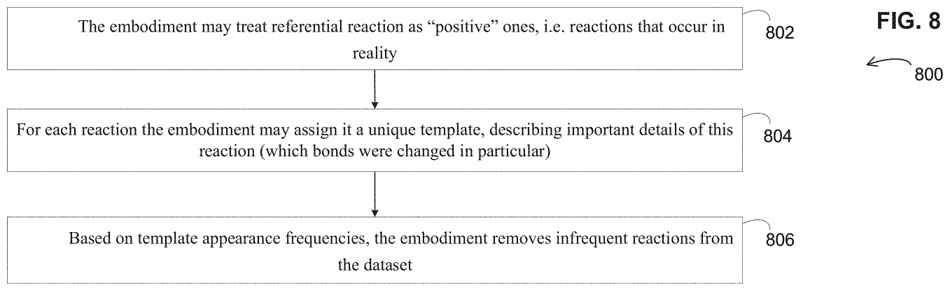

FIG. 8 is a flowchart of steps in an embodiment of a method 800 for constructing a dataset for training a model for providing a probability that a chemical reaction occurs. In the embodiment, the dataset of reactions for training this model is constructed as follows. In step 802, the embodiment may treat referential reaction as “positive” ones, i.e. reactions that occur in reality. In step 804, for each reaction the embodiment may assign it a unique template, describing important details of this reaction (which bonds were changed in particular). In step 806, based on template appearance frequencies, the embodiment removes infrequent reactions from the dataset. This removal prevents invalid reactions from ending up in the dataset.

Training the model may also use “negative” data, that is, reactions determined to have small probability of occurring in practice. Such negative data is synthetical and may be constructed as follows. First, for each referential reaction, the embodiment uses its template to generate a synthetic reaction with the same substrates but a different product. This is a forward or downstream reaction, since the flow goes from substrate to product. This synthetic reaction is a reaction of the same type, which proceeded differently than the original one (e.g., in different place of substrates), and resulted in an alternative product. Then, obtained reactions are marked as “negative” ones, and in this case “forward negative” ones.

The model may be constructed of building blocks, which are well established elements of machine learning models. The embodiment may use Graph Convolutional Neural Networks that work on graph inputs. However, the embodiment may be the first to use this kind of model on a direct representation of a reaction as a single graph. The model learns to predict reaction feasibility based on positive and negative data, by adapting its internal parameters iteratively.

2. Discriminating Chemical Reactions Generated by the System, which do not Match the Distribution of Data Represented by Referential Reactions.

This type of model architecture and training method do not differ extensively from the previous model, but this model may be novel for the following reasons. First, it is directly suited to retrosynthesis problem because of the following conceptual shift during its dataset construction: instead of only using templates found in referential reactions to generate artificial infeasible reactions, the embodiment also utilizes reactions generated by the embodiment itself to construct such negative samples. Second, in comparison to the previous model, this model uses the following additional statistical methods: the embodiment generates reactions using the embodiment's reaction generator and adds reactions that do not match certain statistics of the referential reactions to the negative reactions dataset. The details of computing these statistics are described below regarding “dataset construction.” From the perspective of the generator, the purpose is to maximize scores of ground truth reactions compared to other reactions that could be proposed for the same product, but were not reported in the ground truth dataset.

Dataset construction: The embodiment may use previously described positive and negative data as a base.

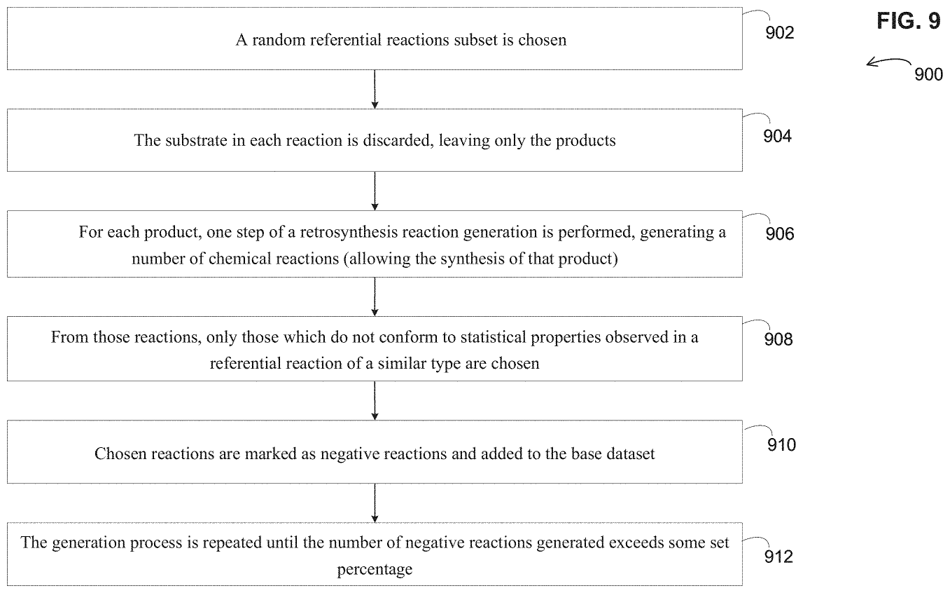

FIG. 9 is a flowchart of steps in an embodiment of a method for creating negative reactions that are backward or upstream in the sense that the flow goes from product to substrate. In the embodiment, a key idea is the addition of additional negative synthetic reactions, created with the following procedure (which is similar to the procedure for reaction generation run during retrosynthesis). In step 902, a random referential reactions subset is chosen. In step 904, the substrate in each reaction is discarded, leaving only the products. In step 906, for each product, one step of a retrosynthesis reaction generation is performed, generating a number of chemical reactions that lead to the synthesis of that product. In step 908, from those reactions, only those which do not conform to statistical properties observed in a referential reaction of a similar type are chosen. In step 910, chosen reactions are marked as negative reactions and added to the base dataset. In step 912, the generation process is repeated until the number of negative reactions generated exceeds some set percentage. This percentage is determined by manually estimating what fraction of generated reactions are usually infeasible. In an embodiment, the number of negative reactions used to train the model is on the same order as the number of positive (“referential”) reactions, which in the embodiment is approximately 1 million positive reactions. Thus, in the embodiment, the model may be trained using approximately 2 million total reactions.

Such backward negative examples represent alternative (different from ground truth) reactions that yield a given compound. Their use in training machine models is not intuitive for chemists because compounds have many possible reactions leading to them, so backward negative examples must contain some false-positives.

Model construction: Proceeds as in the first model. The difference between the first and second models results from the different datasets used during learning, not from a different model structure.

### Chemical Reaction Representation

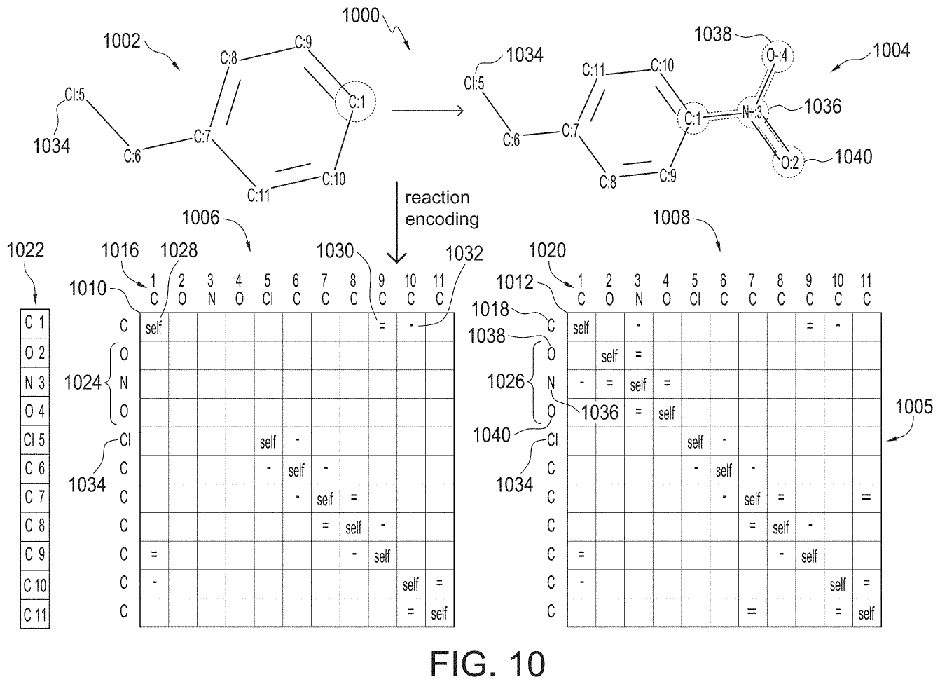

Both models discussed above and used to estimate reaction feasibility are types of Graph Neural Networks, a commonly used machine learning model. However, embodiments may use the following representation, illustrated in FIG. 10, of a chemical reaction as a graph, as input used in training a statistical model.

FIG. 10 is a diagram illustrating an embodiment of a method 1000 for encoding a reaction beginning with a substrate 1002 and yielding a product 1004. In method 1000, the reaction is represented as an undirected multigraph 1005 including a substrate graph 1006 and a product graph 1008. In graph 1005, representing a reaction for machine learning, not all atoms in product 1004 are found in substrate 1002. For example, elements O, N, O 1024 are not found in substrate 1002, but are represented in graph 1006 as shown because they are found in product 1004 (N 1036, O 1038, O 1040). Elements O, N, O 1026 are depicted in product graph 1008. Also, the embodiment may discard some simple compounds, such as water, because their existence in the list of substrates may be deduced implicitly. So, multigraph 1005 is complete and the assumption is and the assumption is that nitrogen and oxygen atoms come from some other compound, for instance NO2. In multigraph 1005, each node (i.e., each atom in top row 1016, 1020, which are the same as each atom in the first column 1022) represents a unique atom in the reaction. An atom that is present in both substrate and the product is represented as a single node. An atom that occurs only in substrates or only in the product is also represented as a single node. In other words, in the embodiment, each atom is represented as a single node and if there is an atom both in substrate and in the product, it is not duplicated and represented as two nodes, but rather represented as a single node. There are two types of edges between atoms: one represents chemical bonds in substrates and the other represents chemical bonds in product. The two types of edges are represented in adjacency matrices 1010 and 1012 of the two separate subgraphs 1006, 1008, respectively. Each entry in a matrix contains a numerical value representing the chemical type of the bond between a pair of atoms (shown symbolically as single bonds (—) or double bonds (═)). The order of the rows and columns corresponds to the labels provided to the atoms in reaction 1004 and mirrored in column 1022 of graph 1005. The order is shown by column 1022 and above each row 1016, 1020, but this listing of the order is optional (although helpful for illustrative purposes). Graph 1005 describes the relationship between atoms before (subgraph 1006) and after (subgraph 1008) the reaction. A model can learn to examine the differences between the substrate subgraph 1006 and product subgraph 1008 to assess the reaction feasibility. To clarify, multigraph 1005 may be used to represent reactions that may be provided to statistical models may take as training input according to one or more embodiments.

In the example shown in FIG. 10, substrate subgraph 1006 is constructed with a row and column for each atom shown in the reaction. Thus, subgraph 1006 includes atoms 1024 that are not shown in the substrate side of the reaction. The atoms may be arranged arbitrarily, but rows 1016 and 1020 and their column orders must be alike. This arrangement results in a diagonal row of identities indicated by “self” where the information on either side of the “self” line is a mirror of the other. Thus, in an embodiment, each matrix 1010, 1012 could be limited to a unique half of the matrix.

### Generating Full Synthesis Path

The former paragraphs described embodiments for how reactions may be proposed for a single target product (“single-step” retrosynthesis). However, embodiments may provide the user with a full path or paths that lead to the target product from simple chemical compounds that are available on the market (“multi-step” retrosynthesis). In embodiments, there are two basic methods of dealing with the multi-step retrosynthesis: In a first, the multi-step retrosynthesis may be solved by recursively proposing reactions leading to compounds that have been proposed for the target molecule and selecting the most promising path due to some heuristic of its value. In a second, the multi-step retrosynthesis task may be solved using a statistical model that learns to propose the most promising reactions, maximizing performance on the referential dataset.

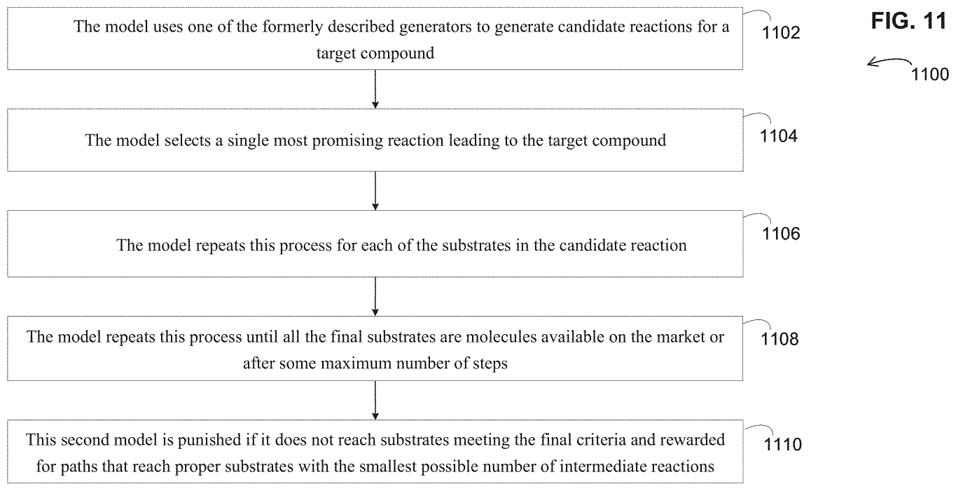

FIG. 11 is a flowchart of steps in an embodiment of a method 1100 for training a model for proposing a synthesis pathway. In step 1102, the model uses one of the formerly described generators to generate candidate reactions for a target compound. In step 1104, the model selects a single most promising reaction leading to the target compound. In step 1106, the model repeats this process for each of the substrates in the candidate reaction. In step 1108, the model repeats this process until all the final substrates are molecules available on the market or after some maximum number of steps. In step 1110, this second model is punished if it does not reach substrates meeting the final criteria and rewarded for paths that reach proper substrates with the smallest possible number of intermediate reactions.

Embodiments of our model for generating the full synthesis path are novel at least because of its combined use of internal modules. 1) Generator using templates and/or deep neural networks. 2) Similarity search to the referential dataset (by molecular fingerprint or trained model). 3) Reaction feasibility estimator. The generator may be used to propose many possibly useful reactions, while the reaction feasibility estimator is used in combination with referential dataset similarity to select the most probable reaction for a target compound.

### The Overall Pathway/Pathways View

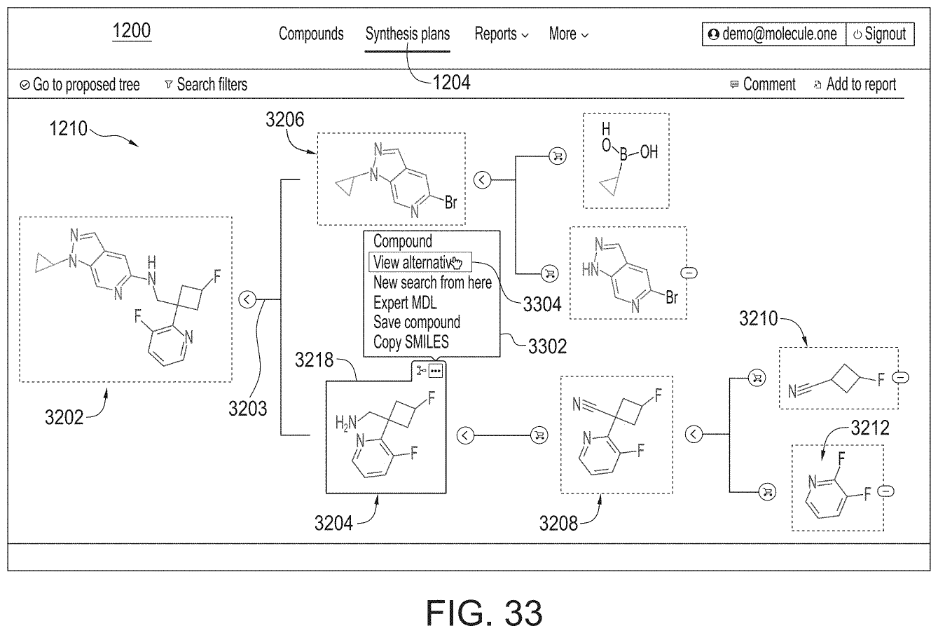

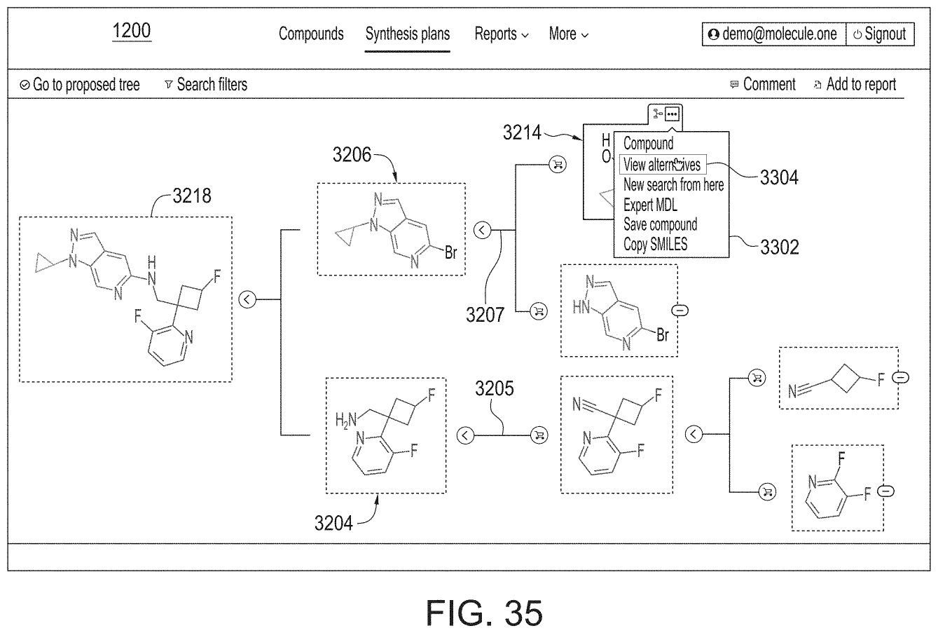

FIG. 12 is a screenshot from an embodiment of a graphical user interface 1200 displaying an embodiment of a multi-step reaction pathway 1210 extracted from a Search Tree. In the embodiment illustrated in FIG. 12, pathway 1210 to target molecule 1228 is presented as a collection of compounds 1212, 1214, 1216, 1218, 1226, 1230, 1232, 1234, with direction arrows (links) 1203, 1207, 1209, 1213, 1217 that represent chemical reactions. Each arrow represents one reaction and goes from the reaction substrate(s) to the reaction product. Thus, many of the compounds are both a substrate and a reaction product. In FIG. 12 the user is provided with proposed synthesis pathway 1210, which was extracted from a search tree, in the order determined by the scoring (also known as the “ranking”) of a number of extracted reaction pathways. The score is the cost of the synthesis pathway as determined in the section Cost Function And The Estimate Of Total Estimated Cost Of Synthesis Pathway. In addition, the section Search Policy (Algorithms Governing The Design Policy) describes a different variant of the cost function that may also be used. For each compound in the synthesis pathway, the user may decide that they want this compound to be synthesized in a different way, or that they want the system to devote more time to this part of analysis. The user may select a compound, e.g., 1226, and the system will redesign the relevant, upstream part of the synthesis pathway, i.e., reactions 1213 and potentially 1217. In FIG. 12, GUI 1200 includes a compound tab 1202, a reaction searches tab 1204 (which is selected, and may be named “synthesis plans”), a saved reactions tab 1206, and a rating tool tab 1208. Reaction searches tab 1204, displays one or more extracted reaction pathways (e.g., pathway 1210), or a status of the reaction search. To assist the user in following a structure or functional group from target molecule 1228 to the source of the structure or functional group, GUI 1200 may color code parts of target molecule 1228 and propagate the color coding to the source of the coded part. For example, target molecule 1228 has color coded section 1220a, 1222a, 1224a. Each of these sections is color coded in upstream reactions to the originating substrate. That is, section 1222a is shown in molecule 1230 as section 1222b, which is the source substrate for section 1220a. For section 1220a, the section is found in molecules 1232, 1223, 1226, 1214, and finally in originating substrate 1218 as section 1220f. A user may use such source information to further inform choices, e.g., choices about which reaction to have the reaction proposing mechanism redesign. In an embodiment of GUI 1200, a button may be displayed near each compound, or the user may be able to click directly on the compound. When the button or compound is selected the system may be requested to perform actions regarding that compound, e.g., to redesign the pathway arriving at, or leading from, that compound. (See, e.g., FIG. 33 and FIG. 35.) Some less important reactions may be hidden (note: in FIG. 12 no reactions are hidden).

Detailed View of a Reaction from the Results

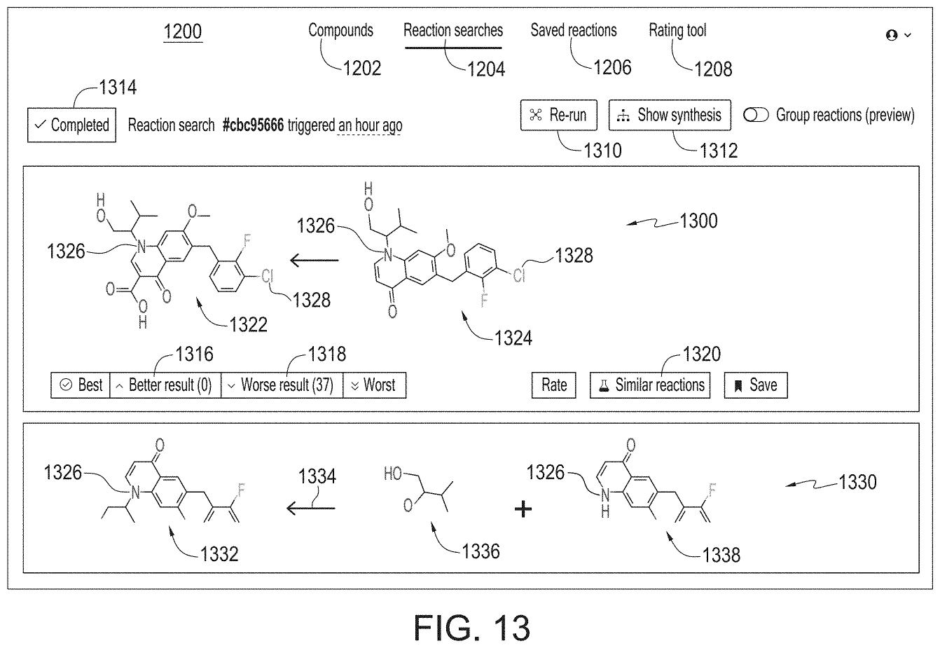

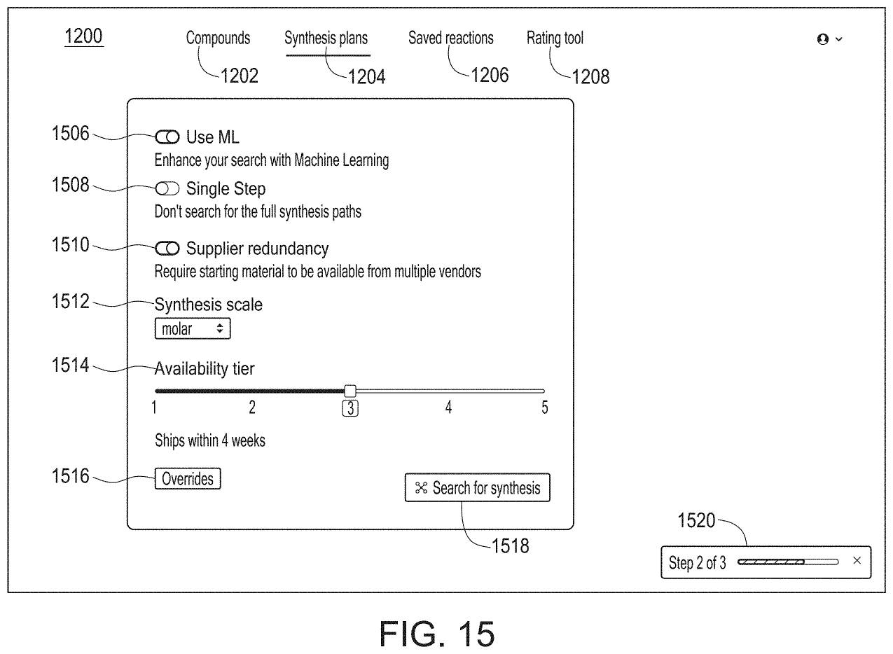

FIG. 13 is a screenshot from an embodiment of GUI 1200 displaying a detailed view of reaction 1300 from a synthesis pathway. In FIG. 13, reactions 1300, 1330 from a synthesis pathway are displayed to the user. GUI 1200 has a status indicator 1314 indicating that searching is completed. GUI 1200 includes options 1310 and 1312 buttons that the user may select to re-run the reaction proposing search 1310 or to show the full synthesis pathway 1312. Using buttons 1316, 1318 the user may navigate between the ranked (better 1316, worse 1318) reaction synthesis pathways that were extracted from the search tree to see other reactions leading to the same product. To see reactions leading to a substrate of the currently viewed reaction, the user may click on the substrate itself. For example, clicking on substrate 1324 shows reaction 1330 leading to that substrate which is shown only in part (1332). By default, reactions leading to the same product are displayed according to the ranking or scoring of the full synthesis pathway they are in (i.e., the ranking is not local for any particular step in a pathway, but global—applying to the entire extracted reaction synthesis pathway—with the being to optimize the entire process and not the single step). The user may choose to look at reactions that are similar to reaction 1300 using button 1320 as determined by a similarity measure, which would then show similar reactions that are similar to reaction 1300. The user may influence the ranking or filter certain reactions by adding appropriate input or making choices in the interface (FIG. 15). Reactions may be grouped using similarity measures in order to make it easier for the user to browse through them. The user may influence how the groups are formed. In an embodiment, the similarity measures are used by a grouping mechanism that groups together reactions that modify the same part or parts of the target molecule. In other embodiments, grouping mechanisms may group based on the type of the reaction (like “deprotection reactions”, “protection reactions”, “carbon-carbon bond forming reactions”, “functional group interconversion” . . . ) or other categories that are well known and meaningful to the chemists. To clarify, similar reactions are reactions that are provided as reference to the reaction in question (so clicking on 1320 yields a screen where references for 1300 are shown); whereas grouping reactions is done for the purpose of easier browsing, not viewing references.” In GUI 1200, a reaction may be color coded so that like elements, functional groups, or structures may be visually tracked by the user. In reaction 1300, in both product 1322 and substrate 1324, N elements 1326 may have the same color. Similarly, CL elements 1328 may have the same color, different from N 1326. In reaction 1330, N elements 1326 may be colored as in reaction 1300 in both product 1332 and substrates 1336, 1338.

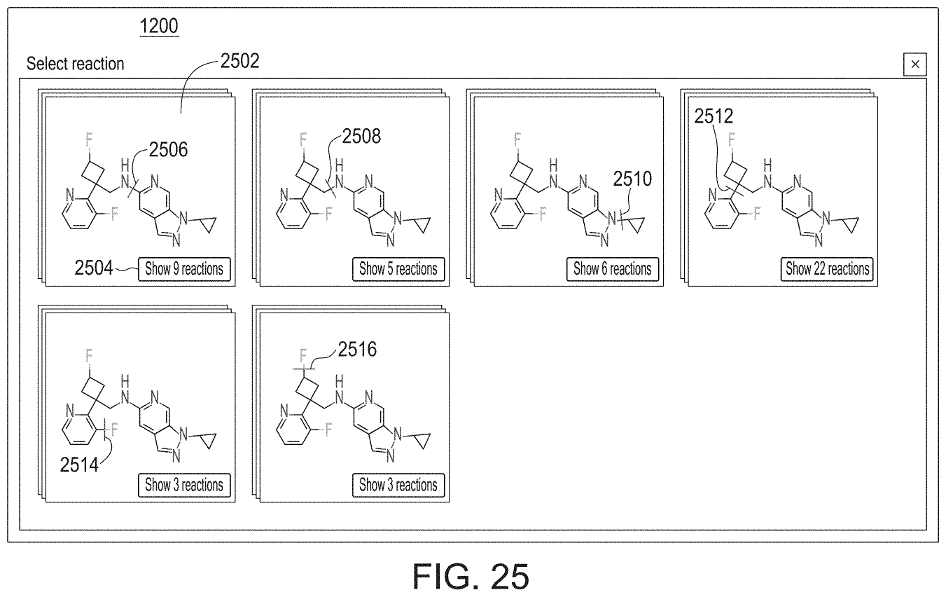

FIG. 25 is a screenshot from an embodiment of a user interface displaying an embodiment of a screen displaying grouped reactions. Each red line (2506, 2508, 2510, 2512, 2514, 2516) marks a bond that is created during each reaction from the corresponding group. The reactions within each group have the creation of that bond in common.

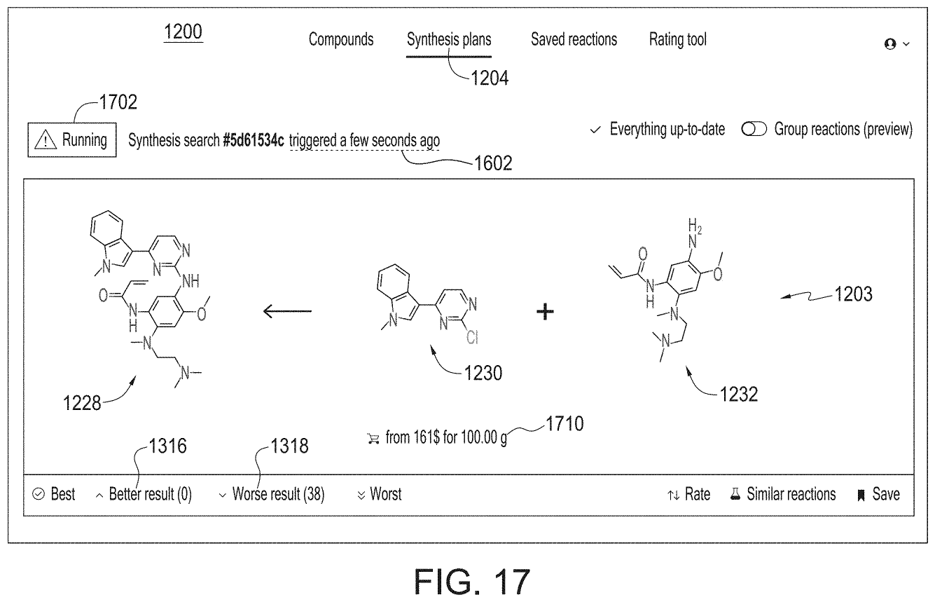

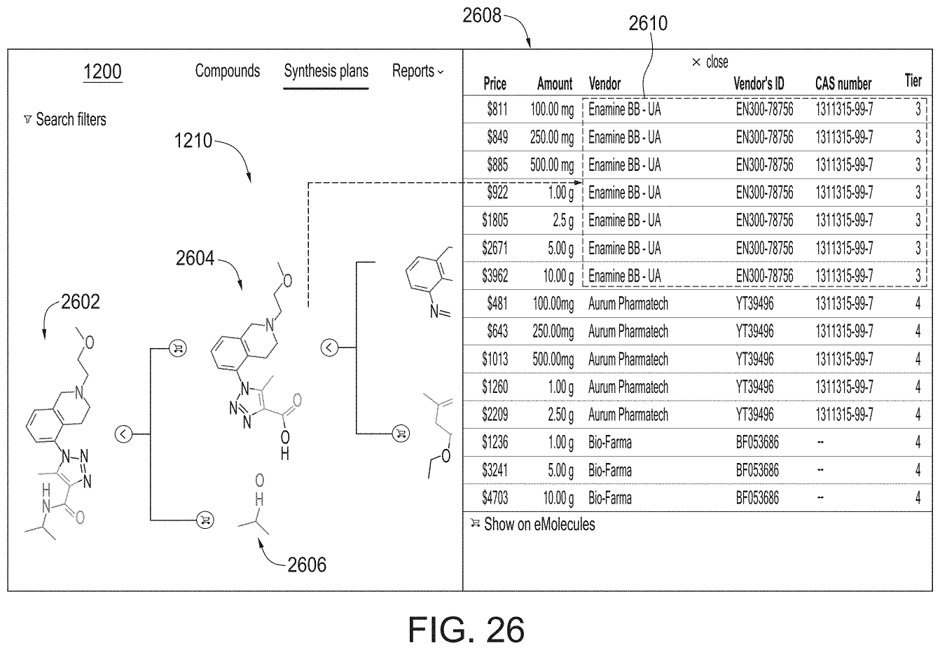

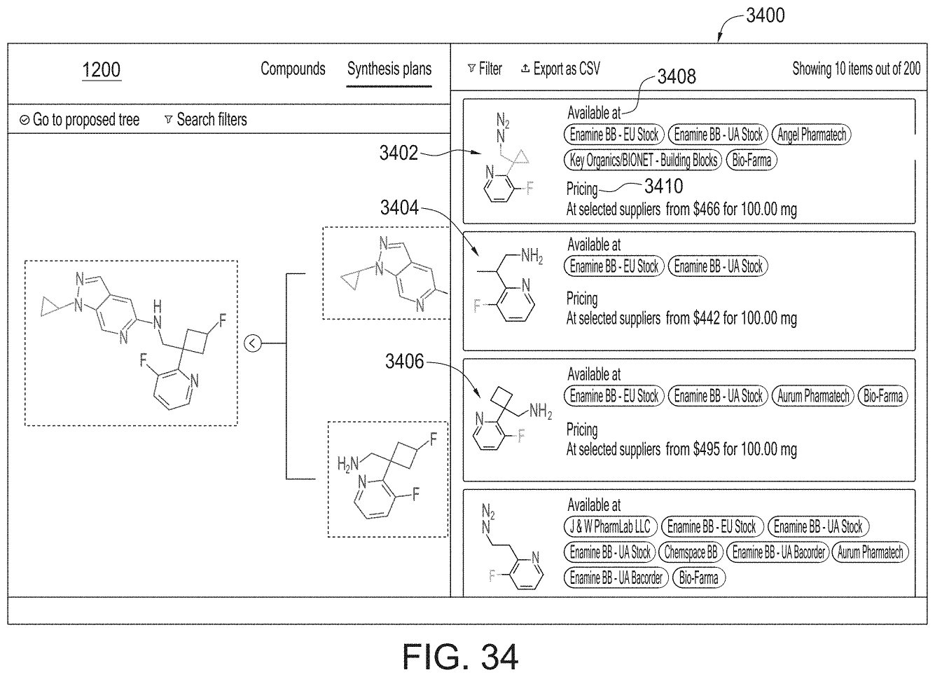

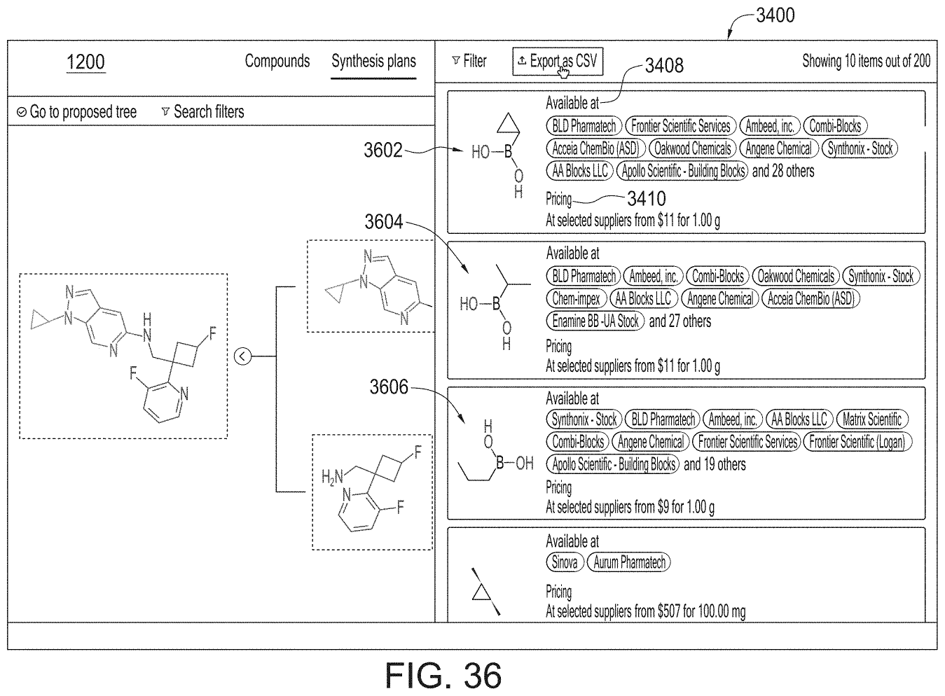

FIG. 26 is a screenshot of user interface 1200 illustrating the display of information 2608 regarding a compound 2604. For each commercially available compound that appears in a designed synthesis pathway, the user may be provided with supporting information that, e.g., may help determine whether it is most cost-effective to buy it or to make it themselves. (See, also FIG. 17-FIG. 19, FIG. 34, FIG. 36, and FIG. 37.) This information may contribute towards a more efficient execution of the synthesis in a lab. In FIG. 26, information 2608 indicates that compound 2604 from reaction synthesis pathway 1210 is available from three different vendors at different prices and amounts. The vendors are ranked according to which tier they belong to. Information 2610 regards the Enamine BB vendor. Enamine BB is listed as a Tier 3 vendor, which in the embodiment means that the compound is in stock. In comparison, tiers 4, and higher, mean that the compound is not in stock. As a result, information 2608 can be used by the user as a constraint on proposed synthesis reaction pathways—the user can require proposed synthesized pathways to require commercially available chemicals to be commercially available and in stock (tier 3 or lower). Additionally, a user-added constraint may be a required number of vendors that have a particular substrate in stock. So, if a user required two or more vendors have compound 2604 in stock before the reaction proposing mechanism would propose it to be a purchased substrate, then compound 2604 would not meet that criteria. As a result, in an embodiment, the reaction proposing mechanism would propose a synthesis pathway the produced compound 2604 from substrates that either met the criteria, or that need to be synthesized themselves. Similar information may be available from compounds 2602 and 2606. In an embodiment, for each vendor, GUI 1200 may provide the ability to proceed to a vendor/procurement site. For every proposed and extracted reaction GUI 1200 may show references to the most similar reactions that can be found in the data the system has access to. Embodiments may be able to search for such references in external data sources or user-provided data.

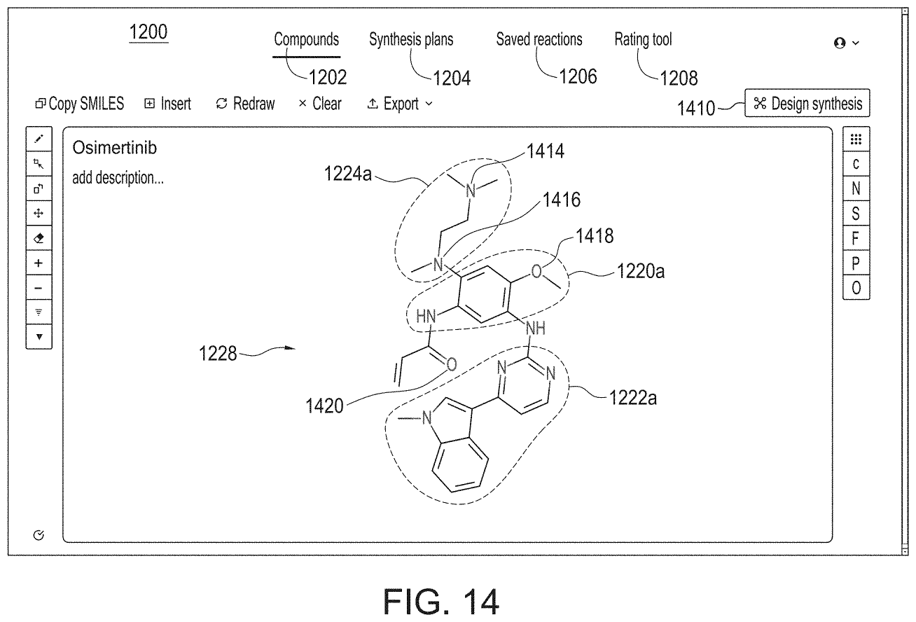

FIG. 14 is a screenshot from an embodiment of a user interface displaying a target compound input screen. In FIG. 14, GUI 1200, within compounds tab 1202 provides the ability for a user to input a target molecule 1228. In the embodiment, the compound may be a known compound imported from an external source (e.g., Osimertinib), or may be created using an embedded molecular editor. In an embodiment, target molecule 1228 may be color coded to assist the use in tracking the synthesis of certain sections. For example, Sections 1220a, 1222a, and 1224a may each have a different coloring. Similarly, elements 1414, 1416 may be similarly colored, and elements 1418, 1420 may be similarly colored. The color coding may assist the user in defining search constraints directly on a molecular structure.

FIG. 15 is a screenshot from an embodiment of a user interface displaying an embodiment of a screen in which a user inputs search parameters. In FIG. 15, within the Synthesis plans tab 1204, a user may be provided with a progress indicator 1520 and options regarding search parameters. For example, option 1506 may provide for the use of machine learning in the reaction proposing mechanism. Option 1508 may provide for limiting the reactions proposed to single step pathways. Option 1510 may provide for requiring that commercially available compounds be available from a certain number of suppliers. Option 1512 may concern the synthesis scale. Option 1514 may further concern suppliers and their ability or timing on shipping. Option 1516 may provide for overrides of standard search parameters, such as, e.g., a standard limit on the number of extracted reaction pathways that are ranked for display. Within the screen, a search for synthesis button 1518 allows the user to launch the system in the search for, and the proposing of, reaction pathways (e.g., pathway 1210).

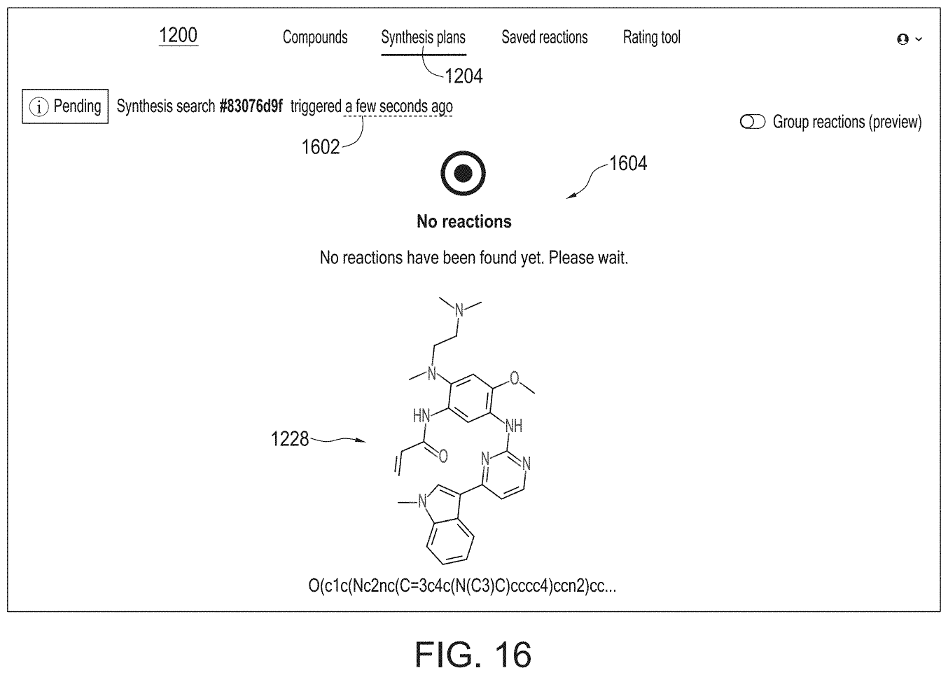

FIG. 16 is a screenshot from an embodiment of a user interface while results are being generated. In FIG. 16 GUI 1200 includes a timer 1602 that provides the time from the initiation of the search for reaction synthesis pathways for target molecule 1228. A reaction results section 1604 changes to reflect the search progress.

FIG. 17 is a screenshot from an embodiment of a user interface displaying detailed views of partial search results. In FIG. 17, GUI 1200 indicates that the search has entered an actively running phase 1702. Reaction results section 1604 has changed to display a proposed reaction 1203 in which target molecule 1228 is the product of a reaction between substrates 1232 and 1230. Price indicator 1710 indicates that substrate 1230 is commercially available and at what price. The lack of a similar price indicator for substrate 1232 may indicate that substrate 1232 may not be commercially available. Ranked results indicators 1316, 1318 show that reaction 1704 is the best of 39 proposed reaction pathways at this point in the computations. The lack of a similar price indicator for substrate 1232 may also be because the system is capable of creating and displaying pathways of reactions where some of the starting materials are not commercially available. That is, the reaction leading to substrate 1232 may be shown when the user clicks on it.

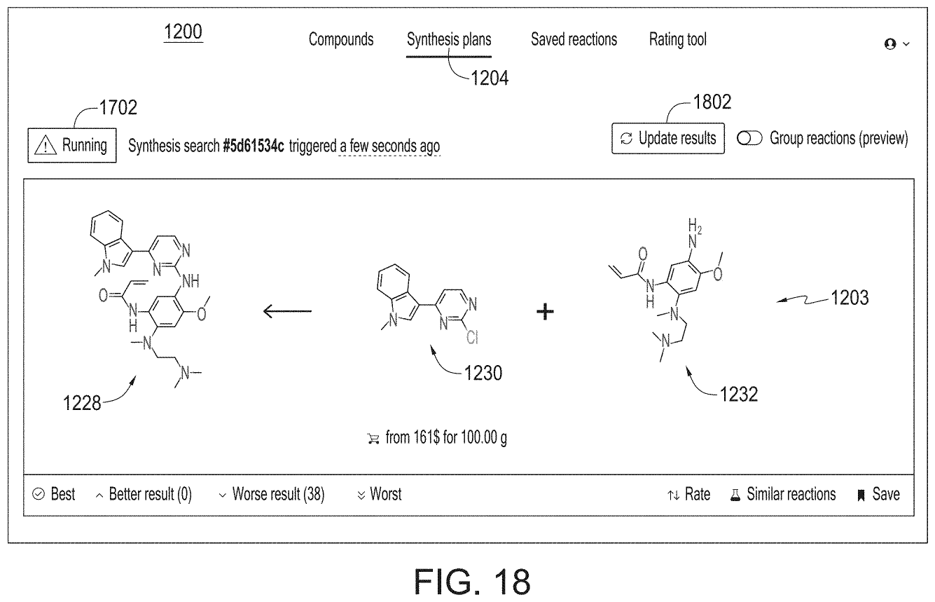

FIG. 18 is a screenshot from an embodiment of a user interface displaying detailed views of partial search results. In FIG. 18, GUI 1200 indicates that the results (39 reaction pathways including pathway 1704) are being updated 1802.

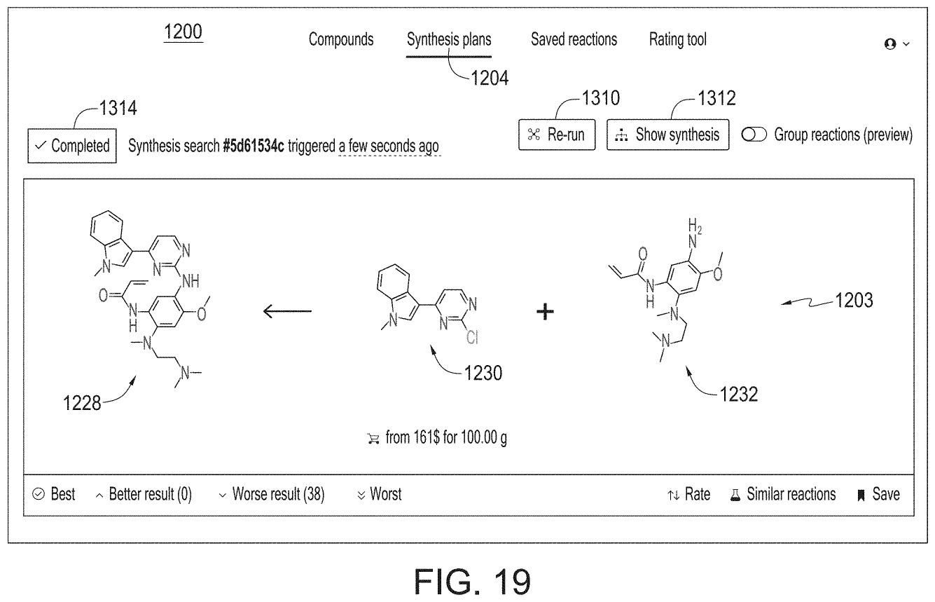

FIG. 19 is a screenshot from an embodiment of a user interface displaying detailed views of finished search results. In FIG. 19, GUI 1200 indicates that the reaction synthesis is complete 1314. As a result, the user is provided with options to re-run the synthesis 1310 (perhaps after changing one or more input parameters), or to show the full reaction synthesis pathway 1312.

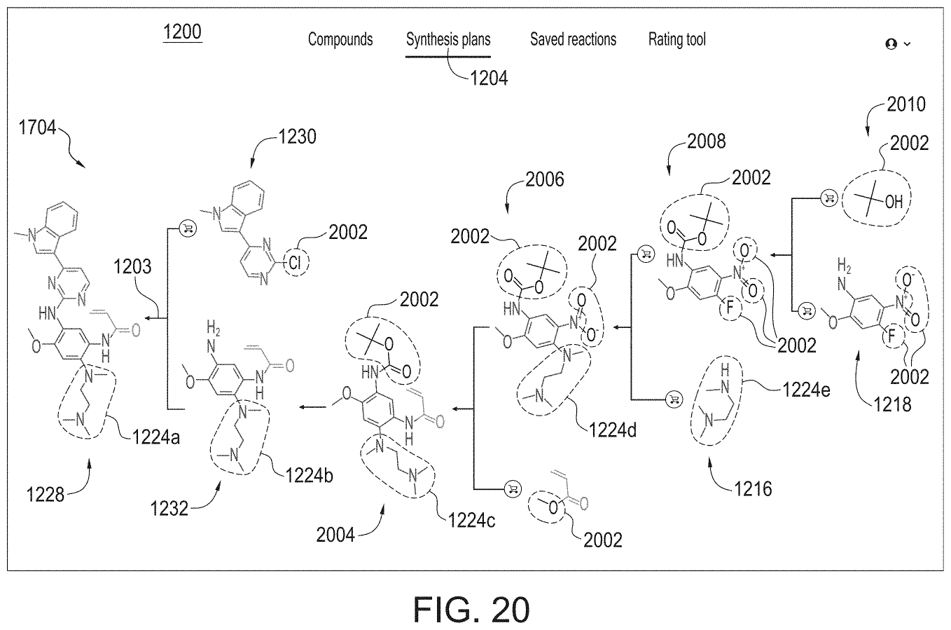

FIG. 20 is a screenshot from an embodiment of a user interface displaying a full synthesis pathway for the results displayed in FIG. 19. In FIG. 20, after show synthesis button 1312 is selected by the user, GUI 1200 displays the full synthesis pathway 1704 for the synthesis of target molecule 1228. In FIG. 20, the cart symbol near a substrate indicates that the substrate is commercially available and, if selected, the cart symbol will provide information regarding the compound. Since a cart symbol is also displayed for compound 2008 and a reaction is proposed to synthesis compound 2008, the display indicates that the system has made a determination that it is more economical to synthesize compound 2008 than it is to purchase compound 2008. Dotted enclosed sections 2002 indicate similarly colored elements that may assist the user in tracking aspects of the pathway between reaction product 1228 and substrates 1216, 1218, 1230, 1232, 2002, 2004, 2006, 2008, and 2010. Although not shown in FIG. 20, other sections of target molecule 1228 may be colored and tracked through reaction pathway 1704 as shown in FIG. 12.

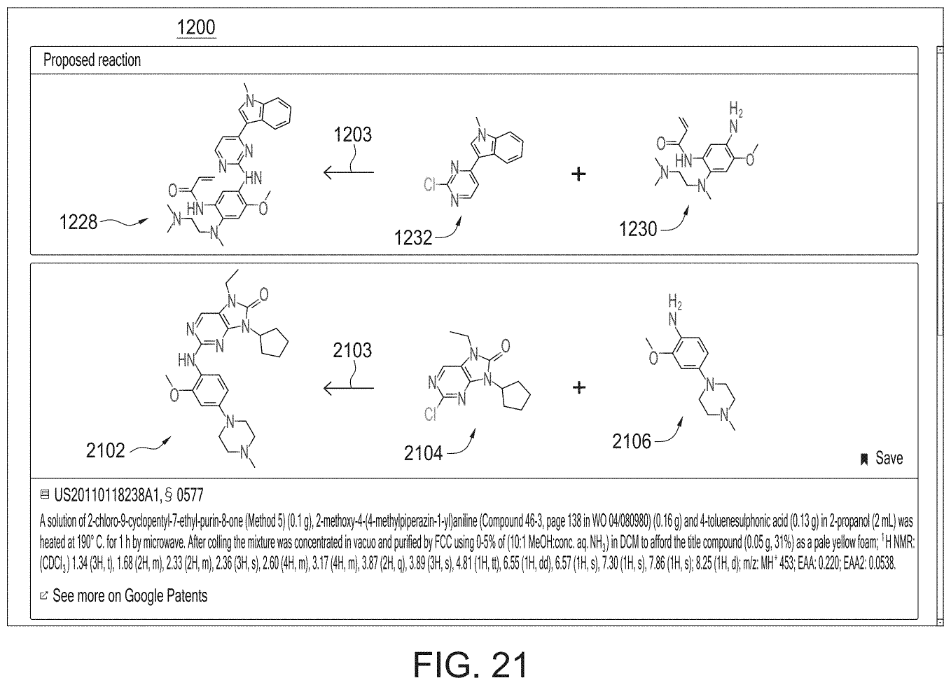

FIG. 21 is a screenshot from an embodiment of a user interface displaying reactions similar to the reaction 1203 of FIG. 19 and FIG. 20, to help the user to execute the reaction in the lab. In FIG. 21, GUI 1200 displays a target molecule 2102 that is the product of a reaction 2103 between substrates 2104 and 2106. In the embodiment, the system determined that target 2102 was similar to target molecule 1228 and reaction 2103 was similar to reaction 1203. Thus it provided reaction 2103 along with its description as supporting information to reaction 1203. Displaying reaction 2103 may help the user execute reaction 1203 because, due to the similarity determination, there is a high probability that the reaction conditions used to execute reaction 2103 will also allow user to execute reaction 1203.

### Planning Synthesis for Multiple Compounds

Currently, according to an embodiment, a reaction proposing mechanism generates a search tree and extracts a reaction pathway from the search tree for the synthesis of a target molecule input by a user. In an embodiment, the user may select a single substrate, e.g., a starting substrate or an intermediate compound in the reaction pathway, and the system may generate an additional group of reactions (downstream from the selected substrate) by replacing the selected compound with a substitute compound chosen by the system from among a group of candidate compounds. In the embodiment, the candidate compounds may all be commercially available compounds as determined by the system searching one or more databases of known compounds. If the selected compound is an intermediate (and not the starting material), the generated pathways are truncated—limited to the downstream reactions—since upstream reactions that lead to the substitute product are no longer necessary. In an embodiment, the user may choose the substitute compound. In either case, the system proposes downstream reactions from the substitute compound.

In an embodiment, an intermediate compound from a reaction pathway may be used in the synthesis of a second target molecule. Thus, two or more synthetic pathways may be proposed, each diverging at a common substrate found at some point in a synthesis pathway. In an embodiment, the second target molecule proposed may be a molecule determined to be as similar as possible to the user's target molecule as determined by a similarity measure, described earlier.

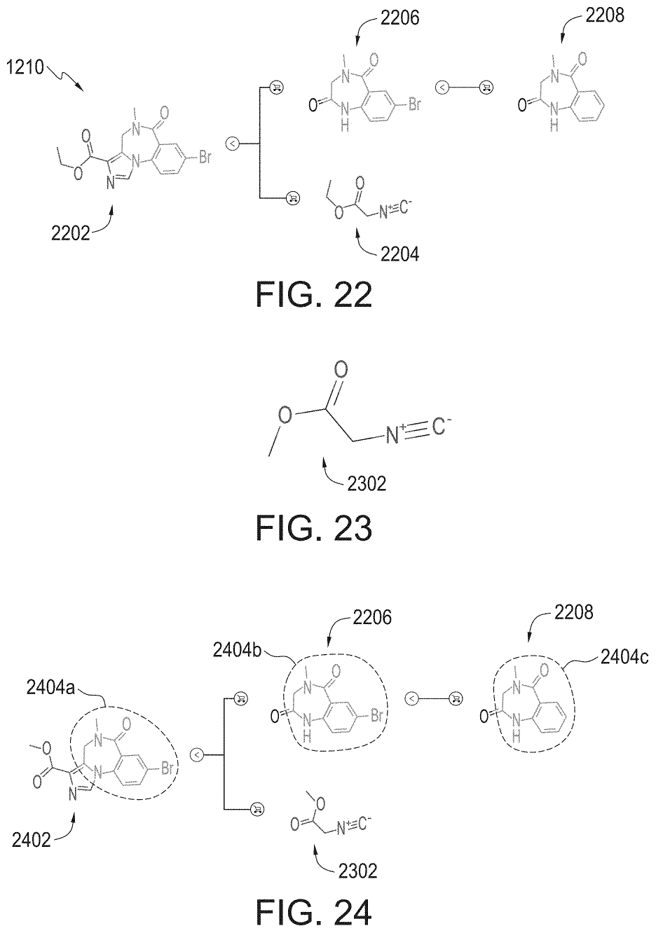

FIG. 22 is an example of a proposed synthesis pathway 2200 generated by an embodiment and producing a user-selected target molecule 2202 from substrates 2204, 2206, and 2208. In the embodiment, the user may select substrate 2204 and request that the system generate a library of alternate compounds. From the generated library, the user, or the system, or both may select substrate 2302 (FIG. 23). Based on new substrate 2302, the system then revises the reactions downstream from compound 2204 to reflect the substitution of compound 2302 for compound 2204. FIG. 24 illustrates the results of the system's revision of the reactions using compound 2302. New reaction product 2402 reflects the use of substituted compound 2302. In an embodiment, a section 2404a of compound 2402 may be colored and tracked through the upstream reactions as 2404b and 2404c to show the origination of part 2404a. Similarly, structure associated with compound 2302 may be similarly colored to show its origin. FIG. 22 through FIG. 24 show two aspects of embodiments. First, the substitution of one substrate for another may result in a different target molecule 2402 versus 2202. Second, a single substrate 2206 may be reacted with two different substrates 2204, 2302 to produce two different target molecules 2202, 2402. In an embodiment of GUI 1200 displaying both first and second target molecules and their associated synthesis pathways, the user may see an advantage in synthesizing intermediate compound 2206 and using compound 2206 to synthesize both the user's target molecule 2202 and the second target molecule 2402. In other words, the user may see an advantage to synthesizing both compounds 2202 and 2402 by executing three reactions instead of four, as the reaction leading from compound 2208 to compound 2206 is the same for both pathways. In an embodiment, the system may provide a listing of commercially available compounds that were proposed as alternatives, and that the user may purchase to synthesize the library.

In an embodiment, alternates to the original substrate may include substrates that may be used such that the downstream reactions in the revised synthesis pathway are not substantially changed from the reactions in the original pathway. That is, the revised synthesis pathway is the same as the original pathway except for changes directly attributable to the structural differences between the original and substituted substrates, and the revised synthesis pathway does not include changes to the types or categories of reactions in the downstream reactions.

In an embodiment, alternate target molecules may be proposed in a ranking determined by how close the alternate target molecule is from the original target molecule. In the embodiment, for each alternate substrate from a library of alternate substrates, the system may generate an alternate target compound. The system may fail to generate an alternate target compound if a reaction in the second synthetic pathway turns out to be infeasible. For each alternate target compound, the system then performs a comparison between the alternate and original target compounds and generates a similarity score. The system then ranks the alternate target compounds according to the similarity score and provides the most similar alternate target compound and associated synthesis pathway, or a ranked listing of alternate target compounds and synthesis pathways, to the user.

In an embodiment, in proposing revised synthesis pathways leading to an alternate target compound, the reaction proposing module employs the same templates that were used to propose the retrosynthesis pathway original target molecule to substrate. Thus, the embodiment uses templates that have already been evaluated and determined to yield feasible results, but they are re-evaluated in the new context. In other words, there may be both feasible and unfeasible reactions yielded by the same template. It is the role of the statistical models to determine feasibility of given reaction.

With reference to FIG. 22, an embodiment planning the synthesis of multiple compounds may be described with reference to a synthesis pathway in which there is one candidate for replacement (compound 2204), only one replacing compound (compound 2302) and only one reaction to be modified (yielding 2202, as in FIG. 22.) In a first series of steps (as discussed with regard to FIG. 6), the system extracts the reaction template from the reaction (yielding 2202, as in FIG. 22), and applies this reaction template to the set of substrates with one of them replaced (2206 and 2302), in the forward, downstream direction. There may be multiple reactions generated as a result.

If, for any of the unchanged substrates in the original reaction, the set of atoms changed during the newly generated reaction is different from the set of atoms changed in the original reaction, the newly generated reaction is discarded. This ensures that the generated reaction modifies (or “takes place”) the same regions of the substrates as the original reaction.

Then, those reactions that are unfeasible according to the statistical models used by the system (and described above) are discarded. Usually, there is at most one reaction remaining. The product of this newly generated reaction is added to the library of compounds that system returns to the user as a compound that may be synthesized.

With a relatively longer synthesis pathway than that of FIG. 22, e.g., synthesis pathway 1704 of FIG. 20, if the candidate compound for replacement is not a substrate in the final reaction of the synthesis pathway (i.e., reaction 1203 of FIG. 20), the above process process described above is repeated for each reaction that leads from the replaced compound to the target compound. For example, if compound 2006 of FIG. 20 were replaced, the above steps would need to be repeated for each reaction between compound 2006 and target molecule 1228.

The process is repeated for each replacing compound. Since there may be millions of such compounds, various optimizations may be utilized. One such optimization, implemented currently in the system, is described as follows. In a first step, the system detects what functional groups in the replaced compound take part in the original reaction. The functional groups are generated, for example, by fragmenting the graph of the replaced compound along the “boring edges” (see the discussion regarding FIG. 6) and interpreting each of the resulting connected component as a functional group. If at least one atom of such functional group is modified during the original reaction, it is interpreted as taking part in the original reaction, and thus, it is necessary for the replacing compound to contain such a functional group.

Then, instead of performing the above steps for each replacing compound, only those replacing compounds are chosen that have all the necessary functional groups for the first modified reactions to take place. This filtering is implemented with a lookup table, where keys are functional group and values are list of compounds that have a given functional group. This process is extremely fast, and in vast majority of cases, reduces the number of commercially available compounds to be considered by at least an order of magnitude.

In an embodiment, the library of generated target compounds may be sorted, filtered, or ranked in many ways. The sorting may be based on commercial availability of the replacing compound, for example price per gram or availability at a certain vendor. The sorting may be based on a compound's estimated ADMET properties, such as toxicity due to reactive functional groups, solubility, partition coefficient etc. (using well established methods). The sorting may be based on the estimated feasibility of the newly generated reaction that lead to a given compound in the library (using statistical models described above). The sorting may be based on the similarity of the generated product to the final product of the original synthesis pathway using, e.g., well established methods, such as ECFP.

FIG. 27 is an illustration of an embodiment of a method for creating negative reactions. In FIG. 27, a reaction 2700 between substrates 2702 and 2704 is shown as having four possible locations 2706, 2708, 2710, 2714 on the benzene ring for bonding substrate 2704 to a carbon atom of substrate 2702 in place of the chlorine atom. FIG. 27 shows “forward” or “downstream” reactions because the arrows indicate direction from substrates to product. Reaction 2706 is considered a positive reaction, because it is a known, referential reaction. In reaction 2706 compound 2704 is joined to compound 2702 at carbon 2714. The location of carbon 2714 is also indicated in compounds 2708, 2710, and 2712 for reference. To create negative reactions 2708, 2710, 2714, compound 2704 is bonded to molecule 2702 at carbon locations that are not known to be feasible, but that are of the same category of reaction. That is, these are the three alternate reactions that are of the same category as the reaction producing compound 2706, where the bond to chlorine is replaced with a bond to a carbon of a benzene ring.

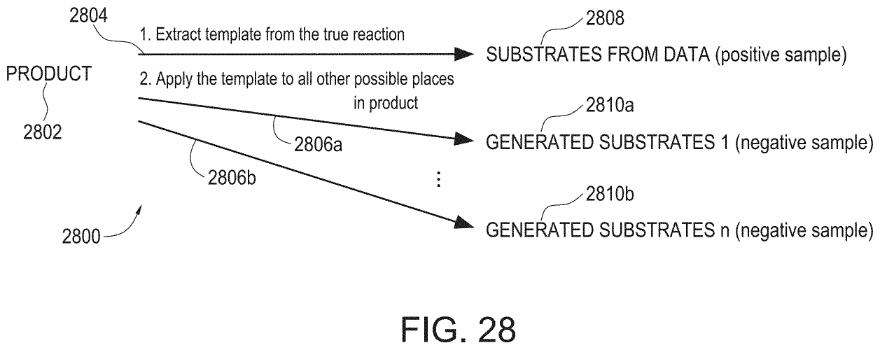

FIG. 28 illustrates an embodiment of a different method for creating negative reactions. FIG. 28 shows “backward” or “upstream” reactions because the arrows indicate direction from product to substrates. In FIG. 28, a product compound 2802 is known to result from a reaction 2804 between substrates 2808. In FIG. 28, the system determines by applying a template (any template, not only the one just extracted) to the product that there are two other possible reactions 2806a, 2806b with combinations of substrates 2810a, 2810b, respectively, that are not found within a database of known reactions. Reactions 2806a, 2806b are then designated as negative reactions. In FIG. 28, two negative reactions are shown but the number of negative reactions is not limited.

In embodiments, both positive and negative reactions are used by the system to train the statistical model to discriminate between feasible and infeasible reactions from the reactions proposed by the reaction generator.

FIG. 29 is a chart showing correlation between an embodiment of a synthetic accessibility score and known scoring methods. An embodiment of a synthetic accessibility score (SAS) is disclosed above—the cost of the synthetic pathway (as in section “Cost Functions And The Estimate Of Total Estimated Cost Of Synthesis Pathway”) is an embodiment of an SAS. In FIG. 29, the M1 Score, Fast M1 Score, M1 Score (distributed), and Fast M1 score (experimental, distributed) are each embodiments of a synthetic accessibility score (SAS) determined by the system for each extracted reaction pathway. An SAS is a measure of the difficulty of executing an extracted synthesis pathway, where a more difficult pathway results in a higher SAS. The SAS is based on the information available to the system, i.e., the extracted reactions, the information associated with each commercially available substrate. Note that in FIG. 29, the Fast M1 score embodiment of the SAS may be used to provide an SAS for tens of thousands of compounds per hour, which is indicative of the number of reactions that need to be processed by the system in order to rank the extracted synthesis pathways. In an embodiment, as SAS measures difficulty of synthesis of a given compound, but isn't tied to a single pathway—as an example, having multiple possible pathways reduces the risk that all of them will fail and thus reduces the difficulty of synthesis.

FIG. 30C is a chart 3000 showing results from the use of an embodiment of a synthetic accessibility score to score synthesis pathways with different numbers of steps in the pathway. FIG. 30A and FIG. 30B are charts 3004, 3002, respectively, showing results from the use of prior art methods of scoring the same reactions scored in FIG. 30C. Each chart lists the number of steps in the reaction pathway across x axis 3014. A comparison of the 2-step path results 3010 from the SAS chart 3000 reactions to the 2-step path results 3012 from the SC score 3002 shows results 3010 to be more tightly grouped. This is true even for 0 steps in the synthesis pathway 3006, 3008, which indicates that the compound is purchased. A comparison of the general results from each chart shows chart 3000 to more clearly reflect the effects of increasing synthesis pathway length.

In embodiments, an SAS provides advantages over previous ways to assess synthetic accessibility because it is based on the extracted synthetic pathway and, using the actual extracted pathway, estimates its execution price, which is then used to calculate and output score. This is found to be more accurate than methods that calculate the score directly from structure using molecular features such as the number of atoms in rings or number of stereocenters.

Because the SAS has access to the extracted pathway, it may account for the set of available starting materials. It's impossible to determine algorithmically the commercial availability of an arbitrary compound knowing just its structure, without access to the databases. That knowledge is important because commercial availability of an intermediate of a synthesis pathway may reduce the numbers of reactions necessary to be executed, and thus reduce complexity of the synthesis significantly.

The fact that in an SAS the cost of the final product is estimated allows the smooth incorporation of the price of starting materials into the final score (a given starting material may have a negligible cost in the case of a small-scale synthesis, but be too expensive when used in a mutli-gram scale synthesis). Usually, in the context of an automatic retrosynthesis, a fixed cutoff is applied (like “only compounds below 100$/g are acceptable starting materials”). That has a problem with the utilization of compounds whose cost is near the threshold—compounds slightly above it are completely disregarded, and the significant cost of compounds just below the threshold is ignored.

Because the SAS has access to the extracted pathway, it may account for the actual reactions that have to be executed. Sometimes, a compound that is significantly different from the desired product may be utilized to quickly synthesize it, and vice versa—a compound that is almost identical to the final compound may be useless for the synthesis of the final compound. For a particular compound, this situation may change as new reactions are discovered. What is also important is that a modification of the compound resulting from one of the reactions in a pathway may enable the utilization of different reactions. Thus it is extremely helpful to actually have access to the synthetic pathway (as methods of calculating an SAS have) if the complexity of the synthesis is to be estimated precisely.

FIG. 37 illustrates these advantages of an SAS. Even though cage structure 3708 (adamantyl group) is regarded as a complex one, target compound 3702 can be easily synthesized in a single step, because a) there is a cheap starting material 3706 that contains this structure; and b) a reaction that utilizes this starting material is feasible. Ignoring any of those factors may lead to incorrect results.

Practical use-cases of an SAS include the following. An SAS score may be used to prioritize structures designed in various phases of the drug discovery pipeline. The prioritized order may be used to decide which should be synthesized first (or synthesized at all). This is important in order to gather information about activity of new structures and make further decisions as quickly as possible. An SAS score may be utilized for multi-objective optimization of the structures generated by in-silico methods; to train the models to generate structures that have desired pharmacological properties and can be easily synthesized.

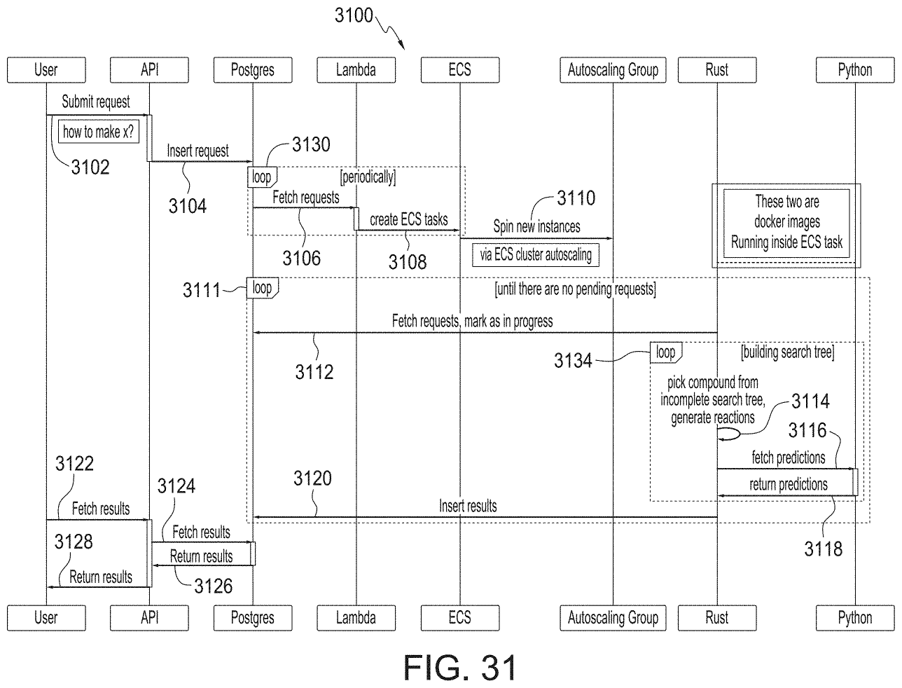

FIG. 31 is a flow chart showing an architecture 3100 for an embodiment of a method for proposing a synthesis pathway. In FIG. 31, in step 3102, the user submits a request for the system to provide a synthesis pathway for a target compound. In step 3104, a Postgres database receives the request from the API layer. In a loop 3130 of steps 3106 and 3108 that is performed periodically, in step 3106, the request fetched from the Postgres database by a Lambda layer, which in step 3108 creates ECS tasks. In step 3110, the ECS layer spins new instances via ECS cluster autoscaling, which are provided by an Autoscaling Group layer. In a step 3111, a loop is performed until there are no pending requests. Loop 3111 includes step 3112 in which requests are fetched from a Postgres database, and marked as “in progress,” as provided to the Rust layer. In step 3134, a loop within loop 3111 builds the search tree with steps 3114 and 3116. In building the search tree, in step 3114, in the Rust layer, a compound is chosen from the incomplete search tree and reactions are generated to synthesize that compound. In step 3116 predictions (or “reaction feasibility estimations”) are fetched from the Python layer by a Rust layer. In step 3118, predictions are returned by the Python layer to the Rust layer. In the embodiment, both the Rust layer and the Python layer are docker images running inside the ECS task. In step 3120, still within loop 3111, results are inserted by the Rust layer into the Postres layer. In step 3122, the user requests results. In step 3124, the API forwards the request for results to the Postgres layer. In step 3126, the Postgres layer returns results 3126 (ranked, extracted synthesis pathways, and other results as described above and displayed to the user via GUI 1200), which, in step 3128, are provided to the user by the API layer. In the architecture shown in FIG. 31, the Postgres (RDS) is for storage and processing queue; the EC2 autoscaling group is used for computation; the API accepts the user query and inserts each compound into the queue; the Lambda layer monitors the queue and creates ECS tasks; the EC2 Autoscaling Group scales according to the number of ECS tasks; task picks up separate compounds to be processed from the queue; and ECS task closes when the queue is empty, at which time the EC2 autoscaling group scales down.

In the embodiment of the method of proposing a synthesis pathway of FIG. 31, data is input into the system in advance of the user interacting with the system. Regarding the reaction data input into the system, the minimum level of information necessary for each reaction in the dataset is a list of substrates and the main product. Bulk access to that reaction data is necessary. Regarding the processing of reaction data. The system includes a chem-inf toolkit (Rust, FIG. 31) and Python (FIG. 31) (PyTorch, RdKit). Regarding the chem-inf toolkit of the Rust layer (FIG. 31), this performs the following functions or steps of an embodiment: normalization of compounds and canonical smiles generation; negative data generation for training statistical classification models; reaction generation and tree search in user application. Furthermore, trained ML models may be embedded in the Rust layer. Regarding the Python layer, this layer performs the following functions or steps of an embodiment: fingerprint calculation for data split (RdKit); reaction graph generation as an input for ML models during training and inference. In an embodiment, the Python layer may be replaced by a ML model embedded in the Rust layer the end user application.

In an embodiment, the reaction proposing mechanism may employ a Template Prior concept. As discussed within this disclosure, embodiments may propose synthesis pathways leading to a target compound. One of the components of the system that both steer the search and participate in a final reaction feasibility estimation is a machine learning model trained on positive and negative reactions (i.e., a dataset of positive (referential) and negative (infeasible) reactions generated according to “Statistical Models For Reaction Feasibility Estimation”) to estimate the feasibility of a reaction, as described within. The output of this machine learning model applied to a particular reaction R (denoted as “M(R)”) estimates the feasibility of R and helps the system choose the most promising reactions. It is also a part of the final reactions/pathway score. It is time consuming to apply the model in every search step. A fast heuristic (the “template prior”) was developed to replace the model during the reaction proposing (also known as “searching”) phase. The use of the fast heuristic “template prior” provides for the decreased use of the model because application of the model may be necessary for only a fraction of all reactions.

In an embodiment, the “template prior” may be defined and created as follows. First, for a reaction R with template T(R), a TemplatePrior(T(R)) is computed as follows:

TemplatePrior(T(R))=(number of positive reactions in the dataset of positive and negative reactions with template T(R))/(number of both positive and negative reactions in the dataset with template T(R)).

Then, the TemplatePrior(T(R)) value is calculated and used instead of the M(R) during the search phase, as a much faster (although less precise) proxy of M(R). The calculation of final results is done using M(R).

In comparisons between the proposing of reaction pathways for a target compound using M(R) values, and using TemplatePrior(T(R)) values, the use of Template Prior values resulted in approximately a 9 times decrease of the total search time on the reference set of test search targets. For ˜95% of test targets using Template Prior, the system was able to find a synthesis path matching the best path found by the original unmodified search that used M(R).

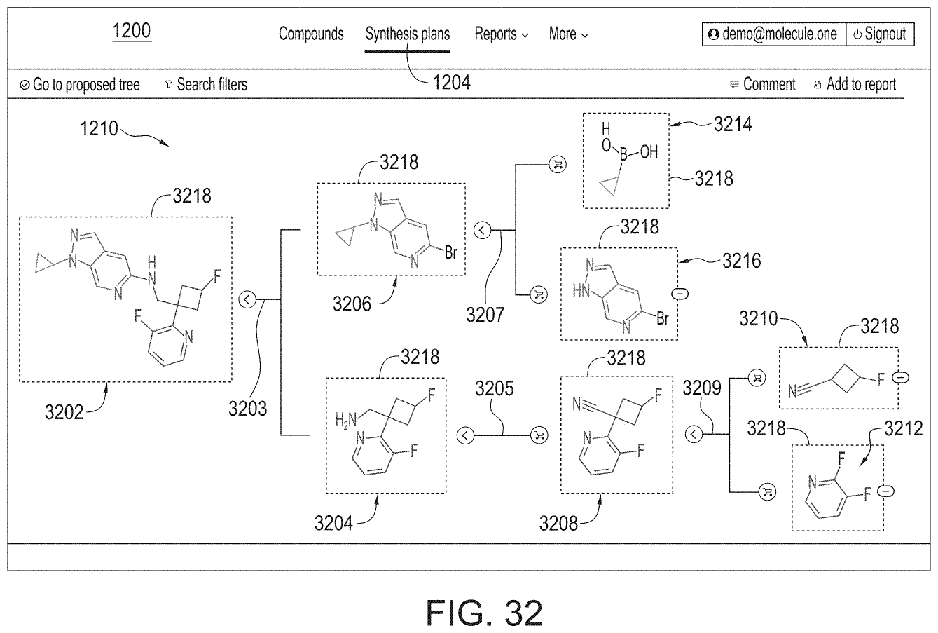

FIG. 32 is a screenshot from an embodiment of a user interface displaying aspects of an embodiment of a method for proposing a synthesis pathway. In FIG. 32, GUI 1200 displays synthesis pathway 1210 in which a target compound 3202 is the product of a sequence of reactions 3203, 3205, 3207, 3209, with starting materials 3210, 3212, 3214, 3216, and intermediates 3204, 3206, 3208. Each compound is shown to be within a region of the GUI indicated by dotted lines 3218. In the embodiment, for each compound, region 3218 may be selected and the use will be provided with options regarding the selected compound.

FIG. 33 depicts the reaction of FIG. 32. In FIG. 33, a user has selected region 3218 associated with compound 3204. In response, GUI 1200 has provided options 3302. In the embodiment, options 3302 include: view alternative 3304, new search from here, expo rt MDL, save compound, and copy SMILES. When the user selects view alternative 3304, GUI 1200 provides the user with compounds that are alternatives to compound 3204, as determined by the system calculating a similarity measure to a library of compounds and provide a ranked listing of the results. In FIG. 34, GUI 1200 displays alternate compound view 3400, including compounds 3402, 3404, 3406, in response to the user's selection. In the embodiment, view 3400 includes additional information regarding each compound, such as sources 3408 and pricing 3410. With such information, the user may opt to select an alternate compound to replace compound 3204. The user may then instruct the system to revise the downstream portion of pathway 1210 to reflect the change from compound 3204 to, e.g., compound 3406. Since compound 3406 is commercially available, the portion of pathway 1210 that is upstream from replaced compound 3204 would be discarded. The system would then revise reaction 3203 to reflect the reaction of compound 3406 with compound 3206 and revise product 3202 accordingly. In this manner, the user may affect the target molecule. The new target molecule and pathway may be saved.

In FIG. 35, a user has selected region 3218 associated with compound 3214. In response, GUI 1200 has provided options 3302. In response to the user selecting view alternative 3304, GUI 1200, in FIG. 36, displays alternate compound view 3400, including compounds 3602, 3604, 3606. Should the user select any of compounds 3602, 3604, 3606 to replace compound 3214, the propagation of that change downstream in synthesis pathway 1210 would result in changes to both compounds 3206 and 3218. Since compound 3214 is a starting material, there are no upstream reactions associated with this change to be discarded.

FIG. 37 is an illustration of an aspect of an embodiment of a method for calculating a synthetic accessibility score (SAS, as calculated according to the section on “Cost Functions And The Estimate Of Total Estimated Cost Of Synthesis Pathway). Factors that affect the SAS include: the number of steps in a synthesis pathway; the certainty of each step (as assessed by the method using AI); the cost of the starting materials; the shape of the synthesis pathway (e.g., convergent or linear); the order of individual reactions within a pathway (riskier reactions are preferably at the beginning of a pathway so their failure has less of an impact).

In a test of an embodiment the SAS, scores were developed for a group of supplied target molecules from a vendor (the large majority of which were considered to have feasible synthesis pathways), and for a group of target molecules from an academic project (the large majority of which were expected to have infeasible synthesis pathways). The test was to determine whether the SASs for the vendor compounds and the SASs for the academic project compounds would reflect the expectation that the vendor compounds were largely feasible and the academic compounds were largely infeasible. In the test, a synthesis pathway was determined for each molecule using an embodiment described above. For the group of vendor compounds, a synthesis pathway could be found for the vast majority of the compounds and the SAS average was approximately 3.5 with a relatively tight distribution. Only a relatively small percentage of the vendor compounds received an SAS of near 10 (which indicates the reaction is infeasible). The feasible compounds from the academic project averaged as SAS of approximately 4 with a distribution almost twice as great. However, the vast majority of the academic compounds received SAS of 10, indicating they were infeasible reactions. Thus, the test correlated to expectations of reaction feasibility.

In FIG. 37, synthesis reaction pathway 3700 includes a target compound 3702 that is the product of a reaction between substrates 3704, 3706. Both substrate 3706 and compound 3702 include a seemingly complex adamantyl moiety 3708. In embodiments, an SAS may be computed for compound 3702, where the SAS is relatively lower than prior art measures of difficulty because the SAS receives information regarding the entire synthesis pathway 3700, including information regarding substrate 3706 and the fact that it is commercially available. In contract, prior art measures are typically based on the reaction product, e.g., 3702, and do not consider the availability of substrates. Thus, a prior art measure may view an adamantyl moiety 3708 and compute an unnecessarily high score (indicating it is difficult to synthesize) for the molecule because the prior art measure does not account for the availability of a starting substrate with the same odd structure 3708.

FIG. 38 is an illustration of an aspect of an embodiment of a method for calculating a synthetic accessibility score (SAS). In FIG. 38, compounds 3802 and 3804 are similar except for the locations of double bonds 3806, 3808, 3810, 3812 and the arrangement of three nitrogen atoms in the five-membered ring. For these compounds, a prior art measure may provide relatively similar synthesis scores because of the compounds' apparent similarities. However, in contrast, an SAS for compound 3802 would be significantly higher than an SAS for compound 3804 because, having an entire synthesis pathway for compound 3802, the method can account for the fact that the synthesis of compound 3802, as reflected in the pathway associated with compound 3802, is significantly more difficult than the synthesis of compound 3804.

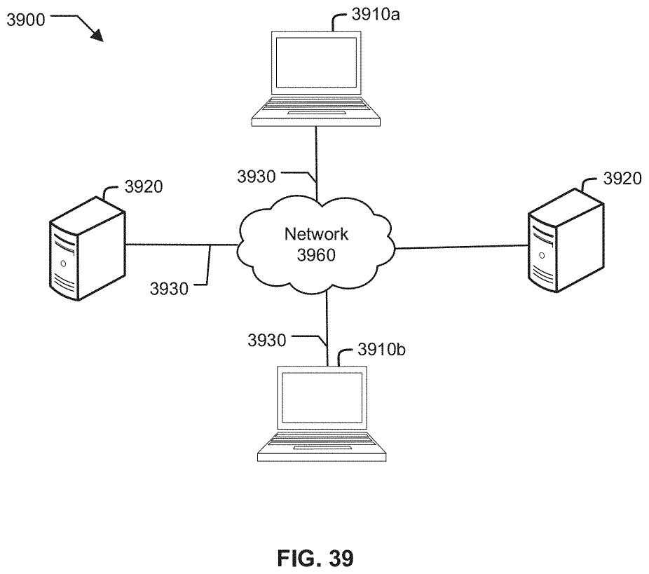

FIG. 39 is an exemplary block diagram depicting an embodiment of system for implement embodiments of methods of the disclosure, e.g., as described with reference to the previous figures, including FIG. 31. In FIG. 39, computer network 3900 includes a number of computing devices 3910a-3910b, and one or more server systems 3920 coupled to a communication network 3960 via a plurality of communication links 3930. Communication network 3960 provides a mechanism for allowing the various components of distributed network 3900 to communicate and exchange information with each other.

Communication network 3960 itself is comprised of one or more interconnected computer systems and communication links. Communication links 3930 may include hardwire links, optical links, satellite or other wireless communications links, wave propagation links, or any other mechanisms for communication of information. Various communication protocols may be used to facilitate communication between the various systems shown in FIG. 39. These communication protocols may include TCP/IP, UDP, HTTP protocols, wireless application protocol (WAP), BLUETOOTH, Zigbee, 802.11, 802.15, 6LoWPAN, LiFi, Google Weave, NFC, GSM, CDMA, other cellular data communication protocols, wireless telephony protocols, Internet telephony, IP telephony, digital voice, voice over broadband (VoBB), broadband telephony, Voice over IP (VoIP), vendor-specific protocols, customized protocols, and others. While in one embodiment, communication network 3960 is the Internet, in other embodiments, communication network 3960 may be any suitable communication network including a local area network (LAN), a wide area network (WAN), a wireless network, a cellular network, a personal area network, an intranet, a private network, a near field communications (NFC) network, a public network, a switched network, a peer-to-peer network, and combinations of these, and the like.

In an embodiment, the server 3920 is not located near a user of a computing device, and is communicated with over a network. In a different embodiment, the server 3920 is a device that a user can carry upon his person, or can keep nearby. In an embodiment, the server 3920 has a large battery to power long distance communications networks such as a cell network or Wi-Fi. The server 3920 communicates with the other components of the system via wired links or via low powered short-range wireless communications such as BLUETOOTH. In an embodiment, one of the other components of the system plays the role of the server, e.g., the PC 3910b.

Distributed computer network 3900 in FIG. 39 is merely illustrative of an embodiment incorporating the embodiments and does not limit the scope of the invention as recited in the claims. One of ordinary skill in the art would recognize other variations, modifications, and alternatives. For example, more than one server system 3920 may be connected to communication network 3960. As another example, a number of computing devices 3910a-3910b may be coupled to communication network 3960 via an access provider (not shown) or via some other server system.

Computing devices 3910a-3910b typically request information from a server system that provides the information. Server systems by definition typically have more computing and storage capacity than these computing devices, which are often such things as portable devices, mobile communications devices, or other computing devices that play the role of a client in a client-server operation. However, a particular computing device may act as both a client and a server depending on whether the computing device is requesting or providing information. Aspects of the embodiments may be embodied using a client-server environment or a cloud-cloud computing environment.

Server 3920 is responsible for receiving information requests from computing devices 3910a-3910b, for performing processing required to satisfy the requests, and for forwarding the results corresponding to the requests back to the requesting computing device. The processing required to satisfy the request may be performed by server system 3920 or may alternatively be delegated to other servers connected to communication network 3960 or to other communications networks. A server 3920 may be located near the computing devices 3910 or may be remote from the computing devices 3910. A server 3920 may be a hub controlling a local enclave of things in an internet of things scenario.

Computing devices 3910a-3910b enable users to access and query information or applications stored by server system 3920. Some example computing devices include portable electronic devices (e.g., mobile communications devices) such as the Apple iPhone®, the Apple iPad®, the Palm Pre™, or any computing device running the Apple iOS™, Android™ OS, Google Chrome OS, Symbian OS®, Windows 10, Windows Mobile® OS, Palm OS® or Palm Web OS™, or any of various operating systems used for Internet of Things (IoT) devices or automotive or other vehicles or Real Time Operating Systems (RTOS), such as the RIOT OS, Windows 10 for IoT, WindRiver VxWorks, Google Brillo, ARM Mbed OS, Embedded Apple iOS and OS X, the Nucleus RTOS, Green Hills Integrity, or Contiki, or any of various Programmable Logic Controller (PLC) or Programmable Automation Controller (PAC) operating systems such as Microware OS-9, VxWorks, QNX Neutrino, FreeRTOS, Micrium μC/OS-II, Micrium μC/OS-III, Windows CE, TI-RTOS, RTEMS. Other operating systems may be used. In a specific embodiment, a “web browser” application executing on a computing device enables users to select, access, retrieve, or query information and/or applications stored by server system 3920. Examples of web browsers include the Android browser provided by Google, the Safari® browser provided by Apple, the Opera Web browser provided by Opera Software, the BlackBerry® browser provided by Research In Motion, the Internet Explorer® and Internet Explorer Mobile browsers provided by Microsoft Corporation, the Firefox® and Firefox for Mobile browsers provided by Mozilla®, and others.

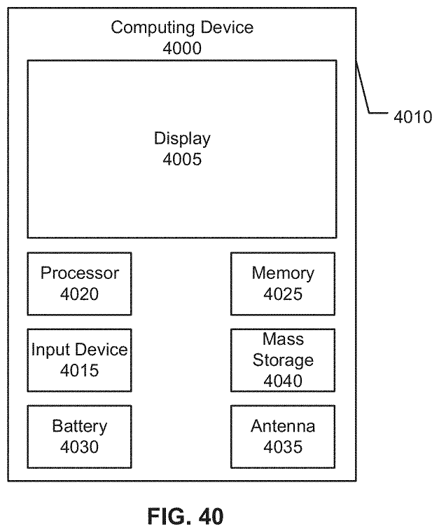

FIG. 40 is an exemplary block diagram depicting a computing device 4000 of an embodiment. Computing device 4000 may be any of the computing devices 3910 from FIG. 39. Computing device 4000 may include a display, screen, or monitor 4005, housing 4010, and input device 4015. Housing 4010 houses familiar computer components, some of which are not shown, such as a processor 4020, memory 4025, battery 4030, speaker, transceiver, antenna 4035, microphone, ports, jacks, connectors, camera, input/output (I/O) controller, display adapter, network interface, mass storage devices 4040, various sensors, and the like.

Input device 4015 may also include a touchscreen (e.g., resistive, surface acoustic wave, capacitive sensing, infrared, optical imaging, dispersive signal, or acoustic pulse recognition), keyboard (e.g., electronic keyboard or physical keyboard), buttons, switches, stylus, or combinations of these.

Mass storage devices 4040 may include flash and other nonvolatile solid-state storage or solid-state drive (SSD), such as a flash drive, flash memory, or USB flash drive. Other examples of mass storage include mass disk drives, floppy disks, magnetic disks, optical disks, magneto-optical disks, fixed disks, hard disks, SD cards, CD-ROMs, recordable CDs, DVDs, recordable DVDs (e.g., DVD-R, DVD+R, DVD-RW, DVD+RW, HD-DVD, or Blu-ray Disc), battery-backed-up volatile memory, tape storage, reader, and other similar media, and combinations of these.

Embodiments may also be used with computer systems having different configurations, e.g., with additional or fewer subsystems. For example, a computer system could include more than one processor (i.e., a multiprocessor system, which may permit parallel processing of information) or a system may include a cache memory. The computer system shown in FIG. 40 is but an example of a computer system suitable for use with the embodiments. Other configurations of subsystems suitable for use with the embodiments will be readily apparent to one of ordinary skill in the art. For example, in a specific implementation, the computing device is a mobile communications device such as a smartphone or tablet computer. Some specific examples of smartphones include the Droid Incredible and Google Nexus One, provided by HTC Corporation, the iPhone or iPad, both provided by Apple, and many others. The computing device may be a laptop or a netbook. In another specific implementation, the computing device is a non-portable computing device such as a desktop computer or workstation.

A computer-implemented or computer-executable version of the program instructions useful to practice the embodiments may be embodied using, stored on, or associated with computer-readable medium. A computer-readable medium may include any medium that participates in providing instructions to one or more processors for execution, such as memory 4025 or mass storage 4040. Such a medium may take many forms including, but not limited to, nonvolatile, volatile, transmission, non-printed, and printed media. Nonvolatile media includes, for example, flash memory, or optical or magnetic disks. Volatile media includes static or dynamic memory, such as cache memory or RAM. Transmission media includes coaxial cables, copper wire, fiber optic lines, and wires arranged in a bus. Transmission media can also take the form of electromagnetic, radio frequency, acoustic, or light waves, such as those generated during radio wave and infrared data communications.

For example, a binary, machine-executable version, of the software useful to practice the embodiments may be stored or reside in RAM or cache memory, or on mass storage device 4040. The source code of this software may also be stored or reside on mass storage device 4040 (e.g., flash drive, hard disk, magnetic disk, tape, or CD-ROM). As a further example, code useful for practicing the embodiments may be transmitted via wires, radio waves, or through a network such as the Internet. In another specific embodiment, a computer program product including a variety of software program code to implement features of the embodiment is provided.

Computer software products may be written in any of various suitable programming languages, such as C, C++, C#, Pascal, Fortran, Perl, Matlab (from MathWorks, www.mathworks.com), SAS, SPSS, JavaScript, CoffeeScript, Objective-C, Swift, Objective-J, Ruby, Rust, Python, Erlang, Lisp, Scala, Clojure, and Java. The computer software product may be an independent application with data input and data display modules. Alternatively, the computer software products may be classes that may be instantiated as distributed objects. The computer software products may also be component software such as Java Beans (from Oracle) or Enterprise Java Beans (EJB from Oracle).

An operating system for the system may be the Android operating system, iPhone OS (i.e., iOS), Symbian, BlackBerry OS, Palm web OS, Bada, MeeGo, Maemo, Limo, or Brew OS. Other examples of operating systems include one of the Microsoft Windows family of operating systems (e.g., Windows 95, 98, Me, Windows NT, Windows 2000, Windows XP, Windows XP x64 Edition, Windows Vista, Windows 10 or other Windows versions, Windows CE, Windows Mobile, Windows Phone, Windows 10 Mobile), Linux, HP-UX, UNIX, Sun OS, Solaris, Mac OS X, Alpha OS, AIX, IRIX32, or IRIX64, or any of various operating systems used for Internet of Things (IoT) devices or automotive or other vehicles or Real Time Operating Systems (RTOS), such as the RIOT OS, Windows 10 for IoT, WindRiver VxWorks, Google Brillo, ARM Mbed OS, Embedded Apple iOS and OS X, the Nucleus RTOS, Green Hills Integrity, or Contiki, or any of various Programmable Logic Controller (PLC) or Programmable Automation Controller (PAC) operating systems such as Microware OS-9, VxWorks, QNX Neutrino, FreeRTOS, Micrium μC/OS-II, Micrium μC/OS-III, Windows CE, TI-RTOS, RTEMS. Other operating systems may be used.

Furthermore, the computer may be connected to a network and may interface to other computers using this network. The network may be an intranet, internet, or the Internet, among others. The network may be a wired network (e.g., using copper), telephone network, packet network, an optical network (e.g., using optical fiber), or a wireless network, or any combination of these. For example, data and other information may be passed between the computer and components (or steps) of a system useful in practicing the embodiments using a wireless network employing a protocol such as Wi-Fi (IEEE standards 802.11, 802.11a, 802.11b, 802.11e, 802.11g, 802.11i, and 802.11n, just to name a few examples), or other protocols, such as BLUETOOTH or NFC or 802.15 or cellular, or communication protocols may include TCP/IP, UDP, HTTP protocols, wireless application protocol (WAP), BLUETOOTH, Zigbee, 802.11, 802.15, 6LoWPAN, LiFi, Google Weave, NFC, GSM, CDMA, other cellular data communication protocols, wireless telephony protocols or the like. For example, signals from a computer may be transferred, at least in part, wirelessly to components or other computers.

The following paragraphs set forth enumerated embodiments.

In embodiment 1 is to a method comprising:

receiving, by a module from at least one software modules, a first molecular structure;

proposing, by a module from the at least one software modules, using the first molecular structure and a model generated by machine learning using known reactions, a first plurality of reactions for synthesizing the first molecular structure, at least one of the first plurality of reactions being created by the module and not retrieved from a database;

extracting, by a module from the at least one software modules, from the first plurality of reactions, at least one first pathway producing the first molecular structure;

predicting, by a module from the at least one software modules, a cost for each extracted first pathway;

ranking, by a module from the at least one software modules, each extracted first pathway according to the predicted cost; and

providing, by a module from the at least one software modules, a listing including each first pathway in an order determined by the ranking.

Embodiment 2 is to the method of embodiment 1 further comprising:

receiving, by the module from the at least one software modules, in addition to the first molecular structure, a constraint on the determining the first plurality of reactions, wherein the module adheres to the constraint in determining the first plurality of reactions.

Embodiment 3 is to the method of embodiment 2, wherein the constraint is defined with reference to the first molecular structure, wherein the module adheres to the constraint in determining the first plurality of reactions.

Embodiment 4 is to the method of embodiment 1 further comprising:

selecting an extracted first pathway;

selecting, from the selected first pathway, a first substrate within the selected first pathway;

comparing, by a module from the at least one software modules, the first substrate to compounds within a database of commercially available compounds;

based on the comparison, choosing, by the module, from the database of commercially available compounds, a second substrate;

substituting, by a module from the at least one software modules, the second substrate for the first substrate in the selected first pathway;

revising, by a module from the at least one software modules, any reaction between the second substrate and the first molecular structure in the selected first pathway to account for the difference between the second substrate and the first substrate, the revising resulting in a second pathway and a change to the first molecular structure such that the result of the second pathway is the second molecular structure; and

associating, by a module from the at least one software modules, the second pathway with the selected first pathway, wherein the providing the listing including each first pathway in an order determined by the ranking includes listing the second pathway with the associated first pathway.

Embodiment 5 is to the method of embodiment 4, wherein: selecting an extracted first pathway includes the user selecting the first pathway; and

selecting, from the selected first pathway, a first substrate that is synthesized by a reaction within the selected first pathway includes a module from the at least one software modules selecting the first substrate.

Embodiment 6 is to the method of embodiment 1, wherein: the proposing, by the module using the first molecular structure and the model generated by machine learning using known reactions, the first plurality of reactions for synthesizing the first molecular structure includes:

creating, by the module, a set of reaction nodes and chemical compound nodes with directional links, the set including a plurality of pathways that yield the first molecular structure; and

the extracting, by the module from the first plurality of reactions, at least one first pathway producing the first molecular structure includes:

extracting, by the module, the at least one first pathway from the set of reaction nodes and chemical compound nodes.

Embodiment 7 is to the method of embodiment 6, wherein the creating, by the module, a set of reaction nodes and chemical compound nodes with directional links, includes beginning with at least the first molecular structure represented by a first chemical compound node in the set and creating, by the module, an expanded set by performing at least one iteration of an expansion, including:

selecting from the set, a chemical compound node to be expanded;

proposing, by the module using the model, at least one additional reaction producing a chemical compound represented by the selected chemical compound node;

adding, by the module, for each proposed additional reaction, a reaction node to the set, and adding a directional link from the reaction node to the selected chemical compound node; and
adding, by the module, for each substrate in each proposed additional reaction, a chemical compound node to the set, and adding a directional link from the added chemical compound node to the reaction node representing the additional reaction.

Embodiment 8 is to the method of embodiment 7, wherein the listing including each first pathway in an order determined by the ranking includes:

displaying, by the module on a computer display, for each first pathway a subset of reaction nodes and chemical compound nodes with directional links extracted from the set of reaction nodes and chemical compound nodes with directional links.

Embodiment 8 is to the method of embodiment 7, wherein, the extracting, by the module from the first plurality of reactions, at least one first pathway producing the first molecular structure includes:

extracting, by the module, the at least one first pathway from the expanded set.

Embodiment 10 is to the method of embodiment 6, wherein the predicting, by the module, a cost for each extracted first pathway includes:

determining, by the module, a probability of success for each reaction node in an extracted pathway by evaluating each reaction node using a statistical model trained to predict reaction feasibility using known reaction data and infeasible reaction data.

Embodiment 11 is to the method of embodiment 10, wherein the infeasible reaction data includes reactions generated by a module from the at least one software modules:

receiving a set of reactions known to occur;

discarding substrates to leave only reaction products;

proposing, using the first molecular structure and the model generated by machine learning using known reactions, for each of the reaction products, a reaction that is a first step in a retrosynthesis of the reaction product;

comparing the generated reactions to the set of reactions known to occur to determine a set of generated reactions that do not conform to properties of the set of reactions known to occur; and adding the set of generated reactions that do not conform to the infeasible reaction data.

Embodiment 12 is to the method of embodiment 1, wherein the proposing, by the module from the at least one software modules, using the first molecular structure and a model generated by machine learning using known reactions, a first plurality of reactions for synthesizing the first molecular structure, includes:

searching, by the module, template graphs of the known reactions for product subgraphs that match a product subgraph of the first molecular structure;

generating, for each matching product subgraph, a proposed set of substrate subgraphs;

removing, by the module, invalid chemical compounds from the proposed set of substrates and the related product subgraph; and

extracting, by the module, a template from each remaining product subgraph and generated set of substrate subgraphs, a reaction template.

Embodiment 13 is to the method of embodiment 1, wherein at least one of the first plurality of reactions for synthesizing the first molecular structure is initially a single step pathway for synthesizing the first molecular structure and the initial single step pathway is expanded to a multi-step pathway by a module from the at least one software modules:

1) designating a substrate from the initial single step pathway as a target molecular structure;
2) proposing, using the target molecular structure and the model, at least one single step pathway for synthesizing the designated target molecular structure; and
3) adding the at least one proposed single step pathway to the first plurality of reactions.

Embodiment 14 is to the method of embodiment 13 further including repeating steps 1-3 for each substrate in the first plurality of reactions until the software module determines that the substrate is found in a database of commercially available compounds, or the software module performs a maximum number of iterations of steps 1-3 for the substrate.

Embodiment 15 is to the method of embodiment 13, wherein an extracted at least one first pathway producing the first molecular structure is a multi-step pathway including a plurality of single step pathways.

Embodiment 16 is to the method of embodiment 13, further comprising ranking an initial subset of the first plurality of reactions, wherein the initial single step pathway is selected from the initial subset of the first plurality of reactions as being a highest-ranked reaction.

Embodiment 17 is to the method of embodiment 1, wherein a subset of the first plurality of reactions includes reactions that become intermediate reactions in one or more of the extracted first pathways.

Embodiment 18 is to the method of embodiment 1, wherein the providing a listing includes providing, by the module from the at least one software modules on a computer monitor, the listing as an interactive display of each first pathway in the order determined by the ranking.

Embodiment 19 is to the method of embodiment 1 further comprising:

providing, by a module from the at least one software modules, for an extracted first pathway, an estimate of difficulty in synthesizing the first molecular structure according to the extracted pathway, the estimate being based at least in part on an analysis, by the module, of each reaction in the extracted first pathway.

Embodiment 20 is to the method of embodiment 19, wherein the estimate is also based on the cost of the extracted first pathway.

Embodiment 21 is to the method of embodiment 1, wherein:

the proposing, by the module from the at least one software modules using the first molecular structure and a model generated by machine learning using known reactions, a first plurality of reactions for synthesizing the first molecular structure includes creating, by the module, an estimate of reaction feasibility for each step in a pathway of the first plurality of reactions; and
the extracting, by the module from the at least one software modules from the first plurality of reactions, at least one first pathway producing the first molecular structure includes using, by the module, the estimates of reaction feasibility in determining which at least one first pathway to extract.

Embodiment 22 is to the method of embodiment 21, wherein the creating, by the model, an estimate of reaction feasibility for each step in a pathway of the first plurality of reactions includes:

creating, by the module using the model, a first estimate of reaction feasibility for each of a first subset of steps in the first plurality of reactions; and

creating, by the module, a second estimate of reaction feasibility for each of a second subset of steps in the first plurality of reactions by: determining a reaction template associated with the step, determining a first number of feasible reactions in a reference dataset that are associated with the same reaction template, determining a second number of infeasible reactions in the reference dataset that are associated with the same reaction template, dividing the first number by a sum of the first and second numbers, the result of the division being the second estimate of reaction feasibility.

Embodiment 23 is to the method of embodiment 1, wherein:

a first module from the at least one software modules performs:
the receiving a first molecular structure; and
the providing a listing including each first pathway in an order determined by the ranking; and a second module from the at least one software modules performs:
the proposing, using the first molecular structure and a model generated by machine learning using known reactions, a first plurality of reactions for synthesizing the first molecular structure, at least one of the first plurality of reactions being created by the module and not retrieved from a database;
the extracting, from the first plurality of reactions, at least one first pathway producing the first molecular structure;
the predicting a cost for each extracted first pathway; and the ranking each extracted first pathway according to the predicted cost.

A system comprising at least one processor and memory with instructions that when executed by the at least one processor cause the system to perform actions according the method of any of embodiments 1-23.

A system comprising at least one processor and memory with instructions that when executed by the at least one processor cause the system to perform actions including:

receiving a first molecular structure;

proposing, using the first molecular structure and a model generated by machine learning using known reactions, a first plurality of reactions for synthesizing the first molecular structure, at least one of the first plurality of reactions being created by the system and not pre-existing in any location accessible by the system;
extracting, from the first plurality of reactions, at least one first pathway producing the first molecular structure;
predicting a cost for each extracted first pathway;
ranking each extracted first pathway according to the predicted cost; and
providing a listing including each first pathway in an order determined by the ranking.

A non-transitory, computer-readable medium comprising instructions that when executed by a processor of a computing device cause the computing device to perform actions according the method of any of embodiments 1-23.

A non-transitory, computer-readable medium comprising instructions that when executed by a processor of a computing device cause the computing device to perform actions including:

receiving a first molecular structure;

proposing, using the first molecular structure and a model generated by machine learning using known reactions, a first plurality of reactions for synthesizing the first molecular structure, at least one of the first plurality of reactions being created by the system and not pre-existing in any location accessible by the system;
extracting, from the first plurality of reactions, at least one first pathway producing the first molecular structure;
predicting a cost for each extracted first pathway;
ranking each extracted first pathway according to the predicted cost; and
providing a listing including each first pathway in an order determined by the ranking.

While the embodiments have been described with regards to particular embodiments, it is recognized that additional variations may be devised without departing from the inventive concept.

The terminology used herein is for the purpose of describing particular embodiments only and is not intended to be limiting of the claimed subject matter. As used herein, the term “and/or” includes any and all combinations of one or more of the associated listed items. As used herein, the singular forms “a,” “an,” and “the” are intended to include the plural forms as well as the singular forms, unless the context clearly indicates otherwise. It will further be understood that the terms “comprises” and/or “comprising,” when used in this specification, specify the presence of states features, steps, operations, elements, and/or components, but do not preclude the present or addition of one or more other features, steps, operations, elements, components, and/or groups thereof.

Unless otherwise defined, all terms (including technical and scientific terms) used herein have the same meaning as commonly understood by one having ordinary skill in the art to which the embodiments belong. It will further be understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

In describing the embodiments, it will be understood that a number of elements, techniques, and steps are disclosed. Each of these has individual benefit and each can also be used in conjunction with one or more, or in some cases all, of the other disclosed elements, or techniques. The specification and claims should be read with the understanding that such combinations are entirely within the scope of the embodiments and the claimed subject matter.

In the description above and throughout, numerous specific details are set forth in order to provide a thorough understanding of an embodiment of this disclosure. It will be evident, however, to one of ordinary skill in the art, that an embodiment may be practiced without these specific details. In other instances, well-known structures and devices are shown in block diagram form to facilitate explanation. The description of the preferred embodiments is not intended to limit the scope of the claims appended hereto. Further, in the methods disclosed herein, various steps are disclosed illustrating some of the functions of an embodiment. These steps are merely examples and are not meant to be limiting in any way. Other steps and functions may be contemplated without departing from this disclosure or the scope of an embodiment.

1. A method comprising:
receiving, by a module from at least one software modules, a first molecular structure;
proposing, by a module from the at least one software modules, using the first molecular structure and a model generated by machine learning using known reactions, a first plurality of reactions for synthesizing the first molecular structure, at least one of the first plurality of reactions being created by the module and not retrieved from a database;
extracting, by a module from the at least one software modules, from the first plurality of reactions, at least one first pathway producing the first molecular structure;
predicting, by a module from the at least one software modules, a cost for each extracted first pathway;
ranking, by a module from the at least one software modules, each extracted first pathway according to the predicted cost; and
providing, by a module from the at least one software modules, a listing including each first pathway in an order determined by the ranking.

2. The method of claim 1 further comprising:
receiving, by the module from the at least one software modules, in addition to the first molecular structure, a constraint on the determining the first plurality of reactions, wherein the module adheres to the constraint in determining the first plurality of reactions.

3. The method of claim 2, wherein the constraint is defined with reference to the first molecular structure, wherein the module adheres to the constraint in determining the first plurality of reactions.

4. The method of claim 1 further comprising:
selecting an extracted first pathway;
selecting, from the selected first pathway, a first substrate within the selected first pathway;
comparing, by a module from the at least one software modules, the first substrate to compounds within a database of commercially available compounds;
based on the comparison, choosing, by the module, from the database of commercially available compounds, a second substrate;
substituting, by a module from the at least one software modules, the second substrate for the first substrate in the selected first pathway;
revising, by a module from the at least one software modules, any reaction between the second substrate and the first molecular structure in the selected first pathway to account for the difference between the second substrate and the first substrate, the revising resulting in a second pathway and a change to the first molecular structure such that the result of the second pathway is the second molecular structure; and
associating, by a module from the at least one software modules, the second pathway with the selected first pathway, wherein the providing the listing including each first pathway in an order determined by the ranking includes listing the second pathway with the associated first pathway.

5. The method of claim 4, wherein:
selecting an extracted first pathway includes the user selecting the first pathway; and
selecting, from the selected first pathway, a first substrate that is synthesized by a reaction within the selected first pathway includes a module from the at least one software modules selecting the first substrate.

6. The method of claim 1, wherein:
the proposing, by the module using the first molecular structure and the model generated by machine learning using known reactions, the first plurality of reactions for synthesizing the first molecular structure includes:
creating, by the module, a set of reaction nodes and chemical compound nodes with directional links, the set including a plurality of pathways that yield the first molecular structure; and
the extracting, by the module from the first plurality of reactions, at least one first pathway producing the first molecular structure includes:
extracting, by the module, the at least one first pathway from the set of reaction nodes and chemical compound nodes.

7. The method of claim 6, wherein the creating, by the module, a set of reaction nodes and chemical compound nodes with directional links, includes beginning with at least the first molecular structure represented by a first chemical compound node in the set and creating, by the module, an expanded set by performing at least one iteration of an expansion, including:
selecting from the set, a chemical compound node to be expanded;
proposing, by the module using the model, at least one additional reaction producing a chemical compound represented by the selected chemical compound node;
adding, by the module, for each proposed additional reaction, a reaction node to the set, and adding a directional link from the reaction node to the selected chemical compound node; and
adding, by the module, for each substrate in each proposed additional reaction, a chemical compound node to the set, and adding a directional link from the added chemical compound node to the reaction node representing the additional reaction.

8. The method of claim 7, wherein the listing including each first pathway in an order determined by the ranking includes:
displaying, by the module on a computer display, for each first pathway a subset of reaction nodes and chemical compound nodes with directional links extracted from the set of reaction nodes and chemical compound nodes with directional links.

9. The method of claim 7, wherein, the extracting, by the module from the first plurality of reactions, at least one first pathway producing the first molecular structure includes:
extracting, by the module, the at least one first pathway from the expanded set.

10. The method of claim 6, wherein the predicting, by the module, a cost for each extracted first pathway includes:
determining, by the module, a probability of success for each reaction node in an extracted pathway by evaluating each reaction node using a statistical model trained to predict reaction feasibility using known reaction data and infeasible reaction data.

11. The method of claim 10, wherein the infeasible reaction data includes reactions generated by a module from the at least one software modules:
receiving a set of reactions known to occur;
discarding substrates to leave only reaction products;
proposing, using the first molecular structure and the model generated by machine learning using known reactions, for each of the reaction products, a reaction that is a first step in a retrosynthesis of the reaction product;
comparing the generated reactions to the set of reactions known to occur to determine a set of generated reactions that do not conform to properties of the set of reactions known to occur; and
adding the set of generated reactions that do not conform to the infeasible reaction data.

12. The method of claim 1, wherein the proposing, by the module from the at least one software modules, using the first molecular structure and a model generated by machine learning using known reactions, a first plurality of reactions for synthesizing the first molecular structure, includes:
searching, by the module, template graphs of the known reactions for product subgraphs that match a product subgraph of the first molecular structure;
generating, for each matching product subgraph, a proposed set of substrate subgraphs;
removing, by the module, invalid chemical compounds from the proposed set of substrates and the related product subgraph; and
extracting, by the module, a template from each remaining product subgraph and generated set of substrate subgraphs, a reaction template.

13. The method of claim 1, wherein at least one of the first plurality of reactions for synthesizing the first molecular structure is initially a single step pathway for synthesizing the first molecular structure and the initial single step pathway is expanded to a multi-step pathway by a module from the at least one software modules:
1) designating a substrate from the initial single step pathway as a target molecular structure;
2) proposing, using the target molecular structure and the model, at least one single step pathway for synthesizing the designated target molecular structure; and
3) adding the at least one proposed single step pathway to the first plurality of reactions.

14. The method of claim 13 further including repeating steps 1-3 for each substrate in the first plurality of reactions until the software module determines that the substrate is found in a database of commercially available compounds, or the software module performs a maximum number of iterations of steps 1-3 for the substrate.

15. The method of claim 13, wherein an extracted at least one first pathway producing the first molecular structure is a multi-step pathway including a plurality of single step pathways.

16. The method of claim 13, further comprising ranking an initial subset of the first plurality of reactions, wherein the initial single step pathway is selected from the initial subset of the first plurality of reactions as being a highest-ranked reaction.

17. The method of claim 1, wherein a subset of the first plurality of reactions includes reactions that become intermediate reactions in one or more of the extracted first pathways.

18. The method of claim 1, wherein the providing a listing includes providing, by the module from the at least one software modules on a computer monitor, the listing as an interactive display of each first pathway in the order determined by the ranking.

19. The method of claim 1 further comprising:
providing, by a module from the at least one software modules, for an extracted first pathway, an estimate of difficulty in synthesizing the first molecular structure according to the extracted pathway, the estimate being based at least in part on an analysis, by the module, of each reaction in the extracted first pathway.

20. The method of claim 19, wherein the estimate is also based on the cost of the extracted first pathway.

21. The method of claim 1, wherein:
the proposing, by the module from the at least one software modules using the first molecular structure and a model generated by machine learning using known reactions, a first plurality of reactions for synthesizing the first molecular structure includes creating, by the module, an estimate of reaction feasibility for each step in a pathway of the first plurality of reactions; and
the extracting, by the module from the at least one software modules from the first plurality of reactions, at least one first pathway producing the first molecular structure includes using, by the module, the estimates of reaction feasibility in determining which at least one first pathway to extract.

22. The method of claim 21, wherein the creating, by the model, an estimate of reaction feasibility for each step in a pathway of the first plurality of reactions includes:
creating, by the module using the model, a first estimate of reaction feasibility for each of a first subset of steps in the first plurality of reactions; and
creating, by the module, a second estimate of reaction feasibility for each of a second subset of steps in the first plurality of reactions by: determining a reaction template associated with the step, determining a first number of feasible reactions in a reference dataset that are associated with the same reaction template, determining a second number of infeasible reactions in the reference dataset that are associated with the same reaction template, dividing the first number by a sum of the first and second numbers, the result of the division being the second estimate of reaction feasibility.

23. The method of claim 1, wherein:
a first module from the at least one software modules performs:
the receiving a first molecular structure; and
the providing a listing including each first pathway in an order determined by the ranking; and
a second module from the at least one software modules performs:
the proposing, using the first molecular structure and a model generated by machine learning using known reactions, a first plurality of reactions for synthesizing the first molecular structure, at least one of the first plurality of reactions being created by the module and not retrieved from a database;
the extracting, from the first plurality of reactions, at least one first pathway producing the first molecular structure;
the predicting a cost for each extracted first pathway; and
the ranking each extracted first pathway according to the predicted cost.

24. A system comprising at least one processor and memory with instructions that when executed by the at least one processor cause the system to perform actions including:
receiving a first molecular structure;
proposing, using the first molecular structure and a model generated by machine learning using known reactions, a first plurality of reactions for synthesizing the first molecular structure, at least one of the first plurality of reactions being created by the system and not pre-existing in any location accessible by the system;
extracting, from the first plurality of reactions, at least one first pathway producing the first molecular structure;
predicting a cost for each extracted first pathway;
ranking each extracted first pathway according to the predicted cost; and
providing a listing including each first pathway in an order determined by the ranking.

25. A non-transitory, computer-readable medium comprising instructions that when executed by a processor of a computing device cause the computing device to perform actions including:
receiving a first molecular structure;
proposing, using the first molecular structure and a model generated by machine learning using known reactions, a first plurality of reactions for synthesizing the first molecular structure, at least one of the first plurality of reactions being created by the system and not pre-existing in any location accessible by the system;
extracting, from the first plurality of reactions, at least one first pathway producing the first molecular structure;
predicting a cost for each extracted first pathway;
ranking each extracted first pathway according to the predicted cost; and
providing a listing including each first pathway in an order determined by the ranking.
